(19) United States
(12) Patent Application Publication
(10) Pub. No.: US 2003/0135177 A1
(43) Pub. Date: Jul. 17, 2003

(54) Absorbent articles containing multi-component core composite and methods of making same

(76) Inventor: Andrew Baker, Alpharetta, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 10/046,554

(22) Filed: Jan. 16, 2002

(51) Int. Cl. A61F 13/15

(52) U.S. Cl. 604/368; 604/378; 604/385.28; 604/386

(57) ABSTRACT

The present invention relates generally to a multi-component absorbent core composite for an absorbent article, and more particularly to a multi-component absorbent core composite comprising at least two absorbent core units having different properties, whereby at least one of the absorbent core units includes a mixture of tow fibers and superabsorbent polymer (SAP) particles. The at least two absorbent core units are associated and arranged with one another to form the multi-component absorbent core composite. Such core composites provide the flexibility of creating precise zoning of particular properties throughout the core, provide improved comfort and fit, and provide less costly means of using expensive core ingredients

### FIELD OF THE INVENTION

The present invention relates generally to a multi-component absorbent core composite for an absorbent article, and more particularly to a multi-component absorbent core composite comprising at least two absorbent core units having different properties, whereby at least one of the core units includes a mixture of tow fibers and superabsorbent polymer (SAP) particles. The at least two absorbent core units are associated with one another to form the multi-component core composite. Such core composites provide the flexibility of creating precise zoning of particular properties throughout the core, provide improved comfort and fit, and provide less costly means of using expensive core ingredients.

### BACKGROUND OF THE INVENTION

Traditionally, disposable absorbent garments such as infant diapers or training pants, adult incontinence products and other such products were constructed with a moisture-impervious outer backing sheet, a moisture-pervious body-contacting inner liner sheet, and a moisture-absorbent core sandwiched between the liner and backing sheets. Much effort has been expended to find cost-effective materials for absorbent cores that display favorable liquid absorbency and retention. Superabsorbent materials in the form of granules, beads, fibers, bits of film, globules, etc., have been favored for such purposes. Such superabsorbent materials generally are polymeric gelling materials that are capable of absorbing and retaining even under moderate pressure large quantities of liquid, such as water and body wastes, relative to their own weight.

The superabsorbent material generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

The ability of a superabsorbent material to absorb liquid typically is dependent upon the form, position, and/or manner in which particles of the superabsorbent are incorporated into the absorbent core. Whenever a particle of the superabsorbent material and absorbent core is wetted, it swells and forms a gel. Gel formation can block liquid transmission into the interior of the absorbent core, a phenomenon called “gel blocking.”Gel blocking prevents liquid from rapidly diffusing or wicking past the “blocking” particles of superabsorbent (e.g., those particles that have swelled and touched an adjacent swelled particle), causing portions of a partially hydrated core to become inaccessible to multiple doses of urine. Further absorption of liquid by the absorbent core must then take place via a diffusion process. This is typically much slower than the rate at which liquid is applied to the core. Gel blocking often leads to leakage from the absorbent article well before all of the absorbent material in the core is fully saturated.

Despite the incidence of gel blocking, superabsorbent materials are commonly incorporated into absorbent cores because they absorb and retain large quantities of liquid, even under load. However, in order for superabsorbent materials to function, the liquid being absorbed in the absorbent structure must be transported to unsaturated superabsorbent material. In other words, the superabsorbent material must be placed in a position to be contacted by liquid. Furthermore, as the superabsorbent material absorbs the liquid it must be allowed to swell. If the superabsorbent material is prevented from swelling, it will cease absorbing liquids.

Adequate absorbency of liquid by the absorbent core at the point of initial liquid contact and rapid distribution of liquid away from this point is necessary to ensure that the absorbent core has sufficient capacity to absorb subsequently deposited liquids. Previously known absorbent cores have thus attempted to absorb quickly and distribute large quantities of liquids throughout the absorbent core while minimizing gel blocking during absorption of multiple doses of liquid.

In general some of the important performance attributes of an absorbent core of a diaper (or any other absorbent garment) are functional capacity, rate of absorption, core stability in use, type of SAP, ratio of fibrous material to SAP, the type and basis weight of glue or tackifying agent used to adhere the SAP to the fibrous material or tissue wrapping, and the basis weight of the core. Absorption under load or AUL is a good measure of functional capacity and the rate at which that absorption occurs. AUL is believed to be a function of both SAP basis weight (mass per unit area) and the composition of SAP used in the composite. Increasing the basis weight decreases the performance/cost ratio of the absorbent core, making them uneconomical. Also, increased basis weights tend to affect the fit and comfort of the garment, as well as impacting the packaging and shipping costs.

It is known to provide absorbent laminates comprised of, for example, an upper layer, a lower layer, and a central fibrous layer containing from 50% to 95% by weight SAP. U.S. Pat. No. 6,068,620, the disclosure of which is incorporated herein by reference in its entirety and in a manner consistent with the present disclosure, discloses that the upper and lower layers are comprised of tissue, airlaid fluff pulp or synthetic nonwoven fibrous layers. The upper and lower layers are said to assist in maintaining the integrity of the core, the laminate layered arrangement is said to minimize gel blocking, and the laminate can be folded in various configurations.

It also is known to provide absorbent cores comprised of differing materials in an attempt to maximize comfort and efficiency of the core. U.S. Pat. No. 5,849,002, the disclosure of which is incorporated by reference herein in its entirety, discloses absorbent cores having three zones: (i) one zone for receiving fluids; (ii) one zone for distributing and storing fluids; and (iii) one zone for preventing leakage. U.S. Pat. No. 5,853,402, the disclosure of which is incorporated by reference herein in its entirety, discloses composite absorbent cores comprising at least an absorbent material and a porous resilient material. Other composite, zoned, or multi-component cores are disclosed in, for example, U.S. Pat. No. 5,425,725, (zones containing pocket regions) U.S. Pat. No. 5,681,300 (blended absorbent core), U.S. Pat. No. 5,882,464 (crimping to join two absorbent structures), U.S. Pat. No. 5,891,120 (varying SAP concentration throughout core), and U.S. Pat. No. 5,983,650 (multiple fiber free SAP pockets in core). The respective disclosures of each of these documents are incorporated by reference herein in their entirety.

### SUMMARY OF THE INVENTION

It would be desirable to provide an absorbent garment having an improved ability to retain fluids and consequently, to prevent leakage. It also would be desirable to provide an absorbent core that includes an increased amount of superabsorbent polymers, but at the same time does not suffer from gel blocking to an appreciable extent. It also would be desirable to provide an absorbent core that has the above mentioned characteristics, and in addition has improved acquisition of fluids, and improved distribution and storage of fluids that insult the core. A further desirable feature would be to make use of expensive core ingredients (like super-SAP), without the attendant increased costs.

It is therefore a feature of an embodiment of the invention to provide an absorbent garment having an improved ability to retain fluids. It is an additional feature of an embodiment of the invention to provide an absorbent garment that includes an absorbent core having SAP particles as a substantial percentage of its basis weight, but at the same time reducing gel blocking, i.e., retaining high SAP efficiency. It is yet a further feature of an embodiment of the invention to provide an absorbent garment that includes an absorbent core having high dry and wet strength for processing and in-use performance. An additional feature of the invention is to provide an absorbent article having specific desired properties in select areas of the absorbent core that is relatively inexpensive to manufacture, that provides the improved properties above, and that is comfortable to wear.

These and other features of the invention can be achieved by an absorbent article including a top sheet, a back sheet and a multi-component absorbent core composite disposed between the top sheet and the back sheet. The multi-component absorbent core composite of the invention is comprised of at least two different absorbent core units having different properties, whereby at least one of the absorbent core units is comprised of a mixture of tow fibers and SAP.

In accordance with an additional feature of an embodiment of the invention, there is provided a method of making an absorbent article that includes providing a top sheet material and a back sheet material. The method also include preparing at least two absorbent core units having different properties, at least one of the absorbent core units being comprised of a mixture of tow fibers and SAP. The method includes arranging the at least two absorbent core units to form a multi-component absorbent core composite, and disposing the multi-component absorbent core composite between the top sheet and the back sheet. Preparing one of the absorbent core units includes dispersing SAP in a central layer that includes tow fibers.

In addition to the foregoing advantages, the absorbent garment having a multi-component absorbent core composite improves the comfort and fit of the garment. Further, due to the thinness of the resulting product, less packaging material is needed for the same amount of product, and shipping and handling costs are lowered. In addition, because highly efficient and expensive materials can be used in specified amounts in specified regions, the costs for producing the absorbent garment may be reduced.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a partially cut-away view of an embodiment of the present invention, shown with elastic members fully stretched in the main portion of the garment;

FIG. 2 is a cross-sectional view of the absorbent garment in FIG. 1 taken along line A-A, illustrating one embodiment for the multi-component absorbent core composite of the invention;

FIG. 3 is a cross-sectional view of a preferred embodiment for the multi-component absorbent core composite of the invention;

FIG. 4 is a top view and a cross-sectional view of an embodiment for the multi-component absorbent core composite of the invention;

FIG. 5 is a top view and a cross-sectional view of an embodiment for the multi-component absorbent core composite of the invention;

FIG. 6 is a top view and a cross-sectional view of an embodiment for the multi-component absorbent core composite of the invention;

FIG. 7 is a top view and a cross-sectional view of an embodiment for the multi-component absorbent core composite of the invention;

FIG. 8 is a top view and a cross-sectional view of an embodiment for the multi-component absorbent core composite of the invention;

FIG. 9 is a top view and a cross-sectional view of an embodiment for the multi-component absorbent core composite of the invention;

FIGS. 10a, 10b, and 10c are top views and cross-sectional views of an embodiment for the multi-component absorbent core composite of the invention;

FIG. 11 is a drawing of one particular embodiment of the core design that illustrates one way to make a core having three zones;

FIG. 12 is a drawing of a particular preferred embodiment of the multiple cores, which has three zones that are zoned in the machine direction. The front and back (zones A and C) are high capacity (low AUL) zones where the pressure due to core expansion is lowest. The center zone B has a high AUL SAP material; and

FIGS. 13a-13f illustrate various configurations for multi-component core composite materials

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of absorbent garments, without limitation, whether disposable or otherwise. The embodiments described herein provide, as an exemplary structure, a diaper for an infant, however this is not intended to limit the claimed invention. The invention will be understood to encompass, without limitation, all classes and types of absorbent garments, including those described herein. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the expression “tow fibers” relates in general to any continuous fiber. Tow fibers typically are used in the manufacture of staple fibers, and preferably are comprised of synthetic thermoplastic polymers. Usually, numerous filaments are produced by melt extrusion of the molten polymer through a multi-orifice spinneret during manufacture of staple fibers from synthetic thermoplastic polymers in order that reasonably high productivity may be achieved. The groups of filaments from a plurality of spinnerets typically are combined into a tow which is then subjected to a drawing operation to impart the desired physical properties to the filaments comprising the tow. It is believed that tow adds surface area to the core, which improves capacity and capillarity as well as surfaces for glue to attach SAP. Tow also is believed to add wet integrity to the core that would otherwise be very poor, as well as add dry integrity that helps with the manufacturing processes.

The present invention relates generally to absorbent articles, and in particular to an absorbent article that contains a top sheet, a back sheet, and a multi-component absorbent core composite disposed between the top sheet and the back sheet. The absorbent core composite of the invention is comprised of at least two different absorbent core units having different properties, whereby at least one of the absorbent core units is comprised of a mixture of tow fibers and SAP.

Throughout this description, the expression “absorbent core unit” or “units” refers to a component of the absorbent core which, if by itself, could function as an absorbent core. Various non-limiting examples of absorbent core units include: a mixture of fibrous material and SAP; SAP enclosed between an upper and lower layer with no fibrous material; a mixture of fibrous material and SAP disposed between an upper layer and a lower layer; an auxiliary layer or layers (fragmented or whole) together with a central fibrous layer containing SAP all disposed between an upper layer and lower layer; and variations, combinations, and mixtures thereof.

The invention also relates in general to a method of making an absorbent article that includes providing a top sheet material and a back sheet material. The method also include preparing at least two absorbent core units having different properties, at least one of the absorbent core units being comprised of a mixture of tow fibers and SAP. The method includes arranging the at least two absorbent core units to form a multi-component absorbent core composite, and disposing the multi-component absorbent core composite between the top sheet and the back sheet. Preparing one of the absorbent core units includes dispersing SAP in a central layer comprising tow fibers.

The absorbent article of the invention preferably has a front waist region, a rear waist region and a crotch region positioned between the front and rear waist regions. The front waist region and rear waist region can be associated with one another to form a waist opening, and two leg openings. Those skilled in the art recognize that “front” and “rear” in the context of the invention denote for clarity purposes only the front and rear of a user, and that the absorbent article could be reversed whereby the previously described “front” portion becomes the rear portion, and vice versa.

Leg elastics preferably are provided along the leg openings for securely holding the leg openings against the thighs of the wearer to improve containment and fit. A fastening system, either resealable or permanent, preferably holds the absorbent article around the wearer's waist. The fastening system assists in associating the front waist region with the rear waist region. A pair of stand-up leg gathers or waist containment flaps may be attached to or formed from the body's side surface of the top sheet.

The preferred embodiments of the absorbent article of the invention include a multi-component absorbent core composite comprising at least two different absorbent core units. At least one of the absorbent core units is comprised of a mixture of tow fibers and SAP. The multi-component absorbent core composite includes one additional absorbent core unit made of any suitable absorbent core materials, and may also include one or more additional components, such as at least one layer selected from an acquisition layer, a distribution layer, an additional fibrous layer containing SAP, a wicking layer, a storage layer, or combinations and fragments of these layers.

Other non-SAP-containing roll good materials such as latex or thermally bonded airlaid fluff pulp, (e.g., roll good available from Walkisoft, Merfin or Fort James), or synthetic spunbonded, carded, or hydro-entangled nonwoven may be positioned above and below the absorbent core. At least one of the absorbent core units preferably contains 50-95% by weight particulate or fibrous SAP and a tow fiber, which preferably is capable of maintaining high SAP efficiency. As described in U.S. Pat. No. 6,068,620, SAP efficiency can be expressed as the ratio of the actual SAP absorbency under load, or AUL (expressed as grams of saline absorbed per gram of SAP in the laminate), and the maximum SAP AUL obtained under ideal conditions of low basis weight where gel blocking does not occur. SAP concentrations of 50-95% provide thinner roll good composites for efficient shaping and handling. High SAP concentrations also provide thinner absorbent cores that can provide new options for product design. The absorbent core units useful in the multi-component absorbent core composites of the invention can be made using either a wet or dry process.

It is particularly preferred in the invention that at least one of the absorbent core units of the multi-component absorbent core composites be comprised of an upper layer, a central layer containing a mixture of tow fibers and SAP, and a lower layer. Forming the absorbent laminate core of the invention with one or more inner layers disposed between an upper and lower layer is believed to decouple key performance attributes of traditional absorbent cores. As recognized by skilled artisans, the various layers of an absorbent core typically are designed with competing interests. A compromise usually is made at the sacrifice of the optimal performance attributes of each of the individual layers. By decoupling the performance attributes of the individual layers, the absorbent cores of the preferred embodiments optimizes the key characteristic performance attributes of each of the inner layers, thereby resulting in overall improved performance over previously known absorbent cores, or absorbent laminates.

Stated more specifically, outer layers of absorbent cores generally are designed for optimal wet/dry strength, liquid acquisition and distribution, as well as SAP containment. The inner layers of absorbent cores generally are designed for optimal absorbency and SAP efficiency. Designers of absorbent cores in the past have had to combine the attributes of the outer and inner layers into a homogeneous composite, often leading to an unacceptable compromise.

Absorbent cores made of tow fibers and SAP typically include a tackifying agent or other type of material to adhere the SAP to the fibers, or to contain the SAP. These cores typically were designed with a single basis weight, a single type of SAP, a single ratio of fiber tow to SAP, a single glue basis weight, and a single glue type. Using different types of glue, SAP, or fiber during manufacture of the core would not be practical.

Varying the absorption capacity as a function of position in the core (e.g., zoned cores) is known. For standard airlaid core forming equipment, however, zoning is problematic. Most of the problems occur when trying to produce low variability zoned cores at high speeds. The problems stem from needing to scarf off material from the areas where low basis weight (absorbency) is desired, and then return that material to the areas where high basis weight (absorbency) is desired.

Most air-formed cores introduce SAP into the pulp stream. This creates a mixture (usually homogeneous) of pulp and SAP that then is laid down into a pocket or screen to create the core. Homogeneous mixtures of SAP and pulp are the most ideal because it prevents gel blocking by increasing the wet core permeability. This makes it difficult, however, to introduce different types of SAP into the core. The standard method of placing SAP in the pulp stream will create a mixture of the two SAPs. While this might be adequate for some types of core designs, it does make it quite difficult to zone one SAP independently of the other SAP and the rest of the core.

Good SAP zoning can be accomplished in conventional airlaid core forming systems. However, this is done by introducing the SAP very close to where the core is finally formed. This means that targeting the SAP in specific locations typically means a highly concentrated layer of SAP. The high concentrations are susceptible to gel-blocking, movement etc. This invention provides a method for introducing different types of SAP in different locations without this common problem.

While not intending on being bound by any theory, the present inventor has found that all of the aforementioned parameters can be varied throughout the cross-sectional area of the core by making a plurality of absorbent core units. For example, one absorbent core unit can be comprised of fibrous material and an expensive “super-SAP.” “Super SAP” in this context simply denotes a SAP material that has superior properties, including, for example, superior AUL, high capacity, high gel strength, high permeability, when compared to SAP materials generally available on the market at that time. This absorbent core unit containing the “super SAP” can be used in only the central insult portions of the multi-component absorbent core composite, while other less expensive absorbent core units can make up the remainder of the composite. Many other possibilities exist, only a few of which are described herein, with reference to the attached drawings.

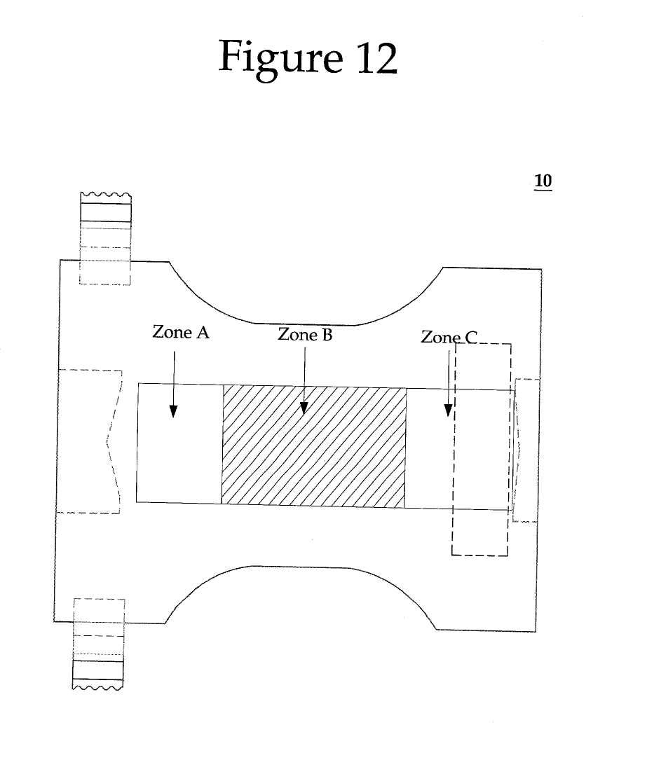

Current SAP technology therefore makes tradeoffs between various properties. It is possible to create a high capacity SAP, but they typically are also low in gel strength. Consequently, it is desirable to introduce the high capacity, low gel strength SAP in areas where capacity is needed, but where the pressure on the core remains relatively low, and to put high AUL SAP in areas where the pressure will tend to be higher. A simple diagram for such a multi component core design is illustrated in FIG. 12.

It is believed by some that AUL correlates with urine leakage, and that higher AUL SAP will reduce leakage. The question why it correlates has eluded some, and the present inventor does not believe that the majority of the pressure on any given SAP particle is due to the weight of the user, although the weight of the user should not be ignored. While not intending on being bound by any theory of operation, however, the present inventor believes that the internal pressures of the weight and volume of the core are much larger contributors to urine leakage. What this means is that the core itself as it swells has a fixed volume to swell into. Once the core has reached that volume it must then swell against the back sheet, top sheet, user's legs, glue bonds, tissue strength, etc., to gain more volume. This is believed to be the primary cause for pressure in diaper cores. As a consequence, the present inventor suggests using high capacity SAP in the ends of the core, (e.g., Zones A and C in FIG. 12), and the high AUL SAP in the crotch region of the core (Zone B in FIG. 12).

Skilled artisans typically design absorbent cores with three properties in mind: fluid acquisition; fluid distribution; and fluid storage. These three properties typically involve tradeoffs or compromises. For example, good distribution typically means high wicking which means small capillary pores. Small pores means relatively slow acquisition. There are other patents that describe the inclusion of wicking layers or storage layers independent of the other layers for this reason. See, for example, U.S. Pat. Nos. 4,798,603, and 5,820,973, the disclosures of which are incorporated by reference herein in their entirety. The present invention is premised in part on the notion that a core can be made up of core components, whereby each component has a different ratio of the aforementioned properties.

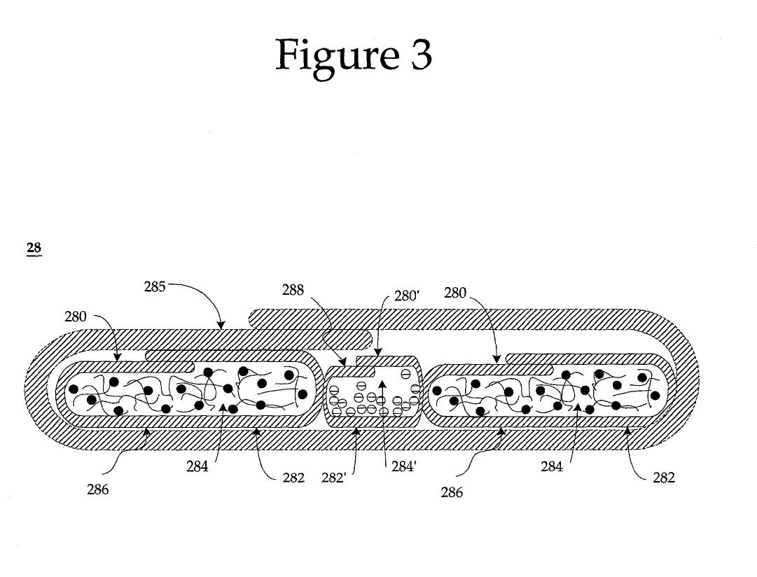

One mechanism to achieve the above varying ratios of properties is to utilize the three components shown in FIG. 3, where the two outer components are designed for high wicking and storage and less for acquisition, while the central component is designed for high acquisition and high storage, but less wicking. A high wicking component can be made with a high wicking tissue outer layer, tow fibers for strength, and SAP particles or fibers or foam. SAP foam itself can make a good acquisition/storage layer. A component having good acquisition and storage properties can be made using high loft materials such as polyurethane foam materials, carded thermalbond nonwovens, Cellulose acetate tow fibers, polyester tow fibers, and the like. Using the guidelines provided herein, those skilled in the art will be capable of designing a suitable multi-component core composite material without undue experimentation.

The invention now will be described with reference to the attached drawings illustrating preferred embodiments of the invention. For clarity, features that appear in more than one Figure have the same reference number in each Figure.

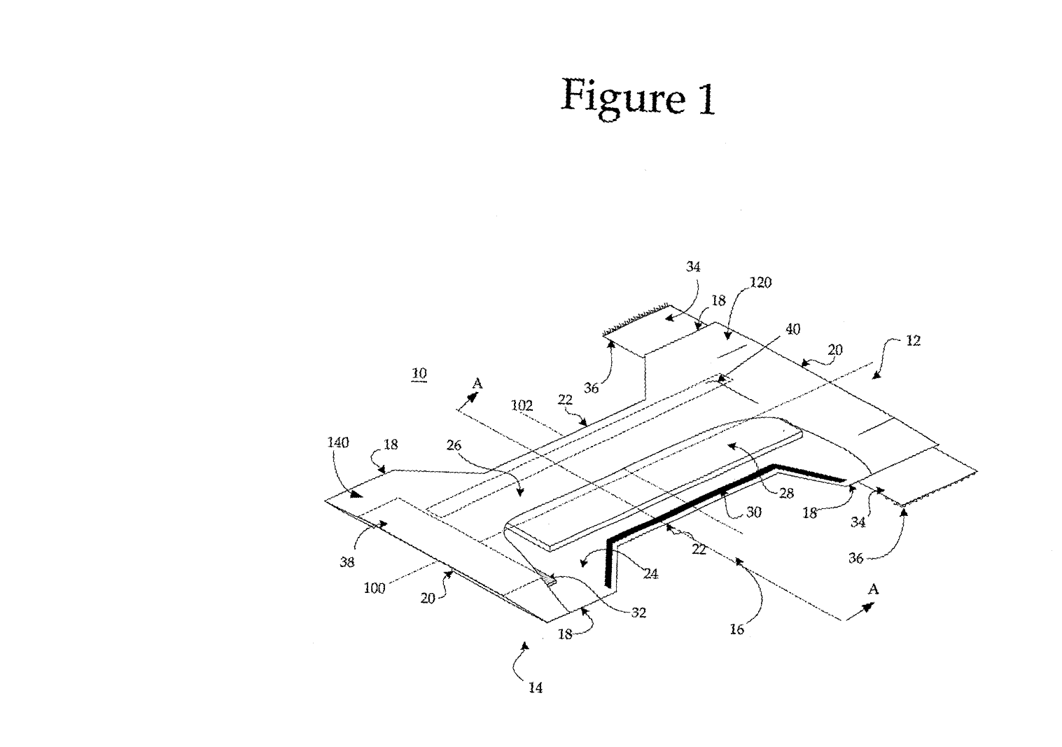

FIG. 1 is a partially cut away depiction of an exemplary embodiment of an absorbent garment 10 (preferably a disposable absorbent garment) of the present invention. The embodiment shown in FIG. 1 is an infant's diaper, however, this depiction is not intended to limit the invention, and those skilled in the art appreciate that the invention covers other types of absorbent articles. For simplicity, however, the invention will be described with reference to an infant's diaper. The garment 10 of FIG. 1 is depicted in a generally flattened position, with the body-facing side facing down, and with the various elastic components depicted in their relaxed condition with the effects of the elastics removed for clarity (when relaxed, the elastics typically cause the surrounding material to gather or “shirr”). In the flattened position, the garment 10 may have a generally hourglass shaped structure, but it may also have any other shape suitable for the given application, such as a rectangular shape, a trapezoidal shape, a “T” shape, and the like.

As used herein, the longitudinal axis 100 of the garment is the dimension of the garment corresponding to the front-to-rear dimension of the user, and the lateral axis 102 of the garment is the dimension corresponding to the side-to-side dimension of the user.

In use, the invention comprises a pant-like garment 10 having a waist-encircling region and a crotch region. The waist-encircling region may comprise a first waist region 12, disposed adjacent to, for example, the back waist region of a wearer's body, and a second waist region 14, disposed adjacent to, for example, the front waist region of a wearer's body. The first and second waist regions 12, 14, may correspond to the front and back of the wearer's body, respectively, depending on whether garment 10 is attached in front of or behind the subject wearer. The first and second waist regions are joined together at or near their lateral edges 18, causing the longitudinally distal edges 20 of the garment 10 to form the perimeter of a waist opening. A crotch region 16 extends between the first and second waist regions 12, 14, and the crotch edges 22 form the perimeter of a pair of leg openings, when the garment 10 is placed on a subject wearer.

The garment 10 preferably comprises a top sheet 24, and a back sheet 26, which may be substantially coterminous with the top sheet 24. When the garment 10 is being worn, the top sheet 24 faces the wearer's body, and the back sheet 26 faces away from the wearer. An absorbent laminate core 28 preferably is disposed between at least a portion of the top sheet 24 the back sheet 26.

An embodiment of the present invention may further comprise various additional features. One or more pairs of elastic gathers 30 may extend adjacent the crotch edges 22. The garment 10 may also comprise one or more waste containment systems, such as inboard standing leg gathers 40, which preferably extend from the second waist region 14 to the first waist region 12 along opposite sides of longitudinal center line 100 (only one standing leg gather system 40 is shown in FIG. 1 for purposes of clarity). One or both of the first and second waist regions 12, 14 may also be equipped with strips of elastic waist foam 32 or other elastically extensible material, which help contract the garment around the wearer's waist, providing improved fit and leakage prevention.

The absorbent garment 10 also preferably includes fastening elements to enable attachment of the first waist region 12 to second waist region 14. Fastening elements preferably include a pair of tabs 34 that extend laterally away from opposite lateral edges 18 of the first waist region 12 of the garment 10. The tabs 34 may comprise an elastically extensible material (not shown), and may be designed to stretch around a wearer's waist to provide improved fit, comfort, and leakage protection. Such elasticized tabs 34 may be used in conjunction with, or in lieu of, waist foam 32, or other elastically extensible materials 32.

At least one fastening mechanism 36 (collectively referred to as “fastener 36”) is attached to each tab 34 for attaching the tab to the second waist region 14, thereby providing the garment 10 with a pant-like shape, and enabling garment 10 to be fixed or otherwise fitted on the wearer. The fasteners 36 may attach to one or more target devices 38 located in the second waist region 14.

Although not shown in the drawings, the absorbent garment 10 may also include grips attached along one of its edges proximal to each tab 34 to enable a caregiver to pull the grips, and not on the ends of the tabs 34, around the wearer and over the target devices 38 to thereby secure the fasteners 36 to the one or more target devices 38.

The various parts of the garment 10 can be attached to one another or associated with one another to form a structure that preferably maintains its shape during the useful life of the garment 10. As used herein, the terms “attached,” “joined,” “associated,” and similar terms encompass configurations whereby a first part is directly joined to a second part by affixing the first part directly to the second part, by indirectly joining the first part to the second part through intermediate members, and by fixing the relative positions of various parts by capturing parts between other parts. Those skilled in the art will appreciate that various methods or combinations of methods may be used to securely join the respective parts of the garment 10 to one another.

The top sheet 24 and back sheet 26 may be constructed from a wide variety of materials known in the art. The invention is not intended to be limited to any specific materials for these components. The top sheet 24 and back sheet can be shaped and sized according to the requirements of each of the various types of absorbent garment, or to accommodate various user sizes. In an embodiment of the invention in which the garment 10 is a diaper or an adult incontinence brief, the combination of top sheet 24 and back sheet 26, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 26 preferably is made from any suitable pliable liquid-impervious material known in the art. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 26 may be pigmented with, for example, titanium dioxide, to provide the garment 10 with a pleasing color or to render the back sheet 26 opaque enough that exudates being contained by the garment 10 are not visible from outside the garment. In addition, the back sheet 26 may be formed in such a manner that it is opaque, for example, by using various inert components in the polymeric film and then biaxially stretching the film. Other back sheet materials will be readily apparent to those skilled in the art. The back sheet 26 preferably has sufficient liquid imperviousness to prevent any leakage of fluids. The required level of liquid imperviousness may vary between different locations on the garment 10.

The back sheet 26 may further comprise separate regions having different properties. In a preferred embodiment, portions of the back sheet 26 are air-permeable to improve the breathability, and therefore comfort, of the garment 10. The different regions may be formed by making the back sheet 26 a composite of different sheet materials, chemical treatment, heat treatment, or other processes or methods known in the art. Some regions of the back sheet 26 may be fluid pervious. In one embodiment of the invention, the back sheet 26 is fluid impervious in the crotch 16, but is fluid pervious in portions of the first and second waist regions 12, 14. The back sheet 26 may also be made from a laminate of overlaid sheets of material.

The moisture-pervious top sheet 24 can be comprised of any suitable relatively liquid-pervious material known in the art that permits passage of liquid there through. Non-woven liner sheet materials are exemplary because such materials readily allow the passage of liquids to the underlying absorbent laminate core 28. Examples of suitable liner sheet materials include non-woven spunbond or carded webs of polypropylene, polyethylene, nylon, polyester and blends of these materials.

The back sheet 26 may be covered with a fibrous, nonwoven fabric such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 26 may comprise three panels wherein a central poly back sheet panel is positioned closest to absorbent laminate core 28 while outboard non-woven breathable side back sheet panels are attached to the side edges of the central poly back sheet panel. Alternatively, the back sheet 26 may be formed from microporous poly coverstock for added breathability.

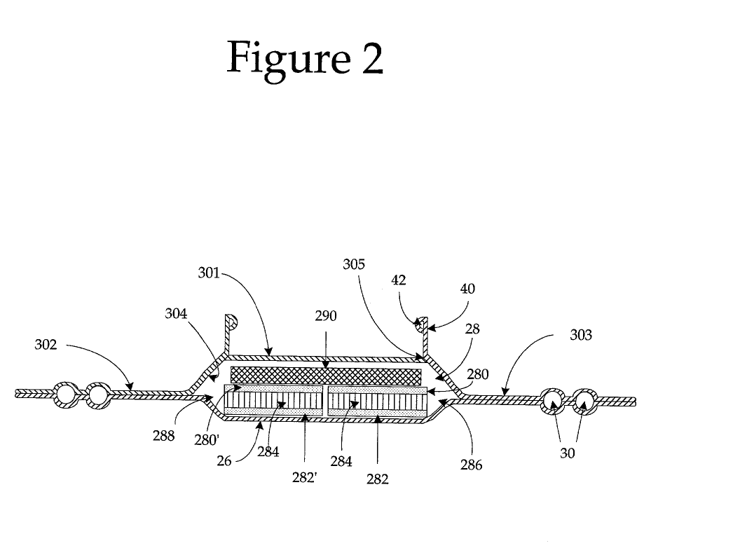

As illustrated in more detail in FIG. 2, the top sheet 24 may be formed of three separate portions or panels. Those skilled in the art will recognize, however, that top sheet 24 need not be made of three separate panels, and that it may be comprised of one unitary item. A first top sheet panel 301 may comprise a central top sheet panel formed from preferably a liquid-pervious material that is either hydrophobic or hydrophilic. The central top sheet panel 301 may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel 301 is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet 301 panel preferably extends from substantially the second waist region 14 to the first waist region 12, or a portion thereof.

The second and third top sheet panels 302, 303 (e.g., outer top sheet panels), in this alternative embodiment may be positioned laterally outside of the central top sheet panel 301. The outer top sheet panels 302, 303 are preferably substantially liquid-impervious and hydrophobic, preferably at least in the crotch area. The outer edges of the outer top sheet panels may substantially follow the corresponding outer perimeter of the back sheet 26. The material for the outer top sheet portions or panels is preferably polypropylene and can be woven, non-woven, spunbonded, carded or the like, depending on the application.

The inner edges 304 (FIG. 2) of the outer top sheet portions or panels 302, 303 preferably are attached by, e.g., an adhesive, to the outer edges 305 of the inner top sheet portion or panel 301. At the point of connection with the outer edges 305 of the inner top sheet portion or panel 301, the inner edges 304 of the outer top sheet portions or panels 302, 303 extend upwardly to form waste containment flaps 40. The waste containment flaps 40 preferably are formed of the same material as the outer top sheet portions or panels 302, 303, as in the embodiment shown. They are preferably an extension of the outer top sheet portions or panels 302, 303.

The waste containment flaps 40 may be treated with a suitable surfactant to modify their hydrophobicity/hydrophilicity as desired, and they may be treated with skin wellness ingredients to reduce skin irritation. Alternatively, the waste containment flaps 40 may be formed as separate elements and then attached to the body side liner. In this alternative embodiment, the central top sheet portion or panel 301 may extend past the connection point with the waste containment flaps 40, and even extend to the periphery of the back sheet 26.

The waste containment flaps 40 preferably include a portion that folds over onto itself to form a small enclosure. At least one, and depending on the size of the enclosure sometimes more than one, elastic member 42 may be secured in the enclosure in a stretched condition. It is known in the art that when the flap elastic 42 attempts to assume the relaxed, unstretched condition, the waste containment flaps 40 rise above the surface of the central top sheet portion or panel 301.

The top sheet 24 (as well as top sheet portions 301, 302, 303) may be made of any suitable relatively liquid-pervious material currently known in the art or later discovered that permits passage of a liquid therethrough. Examples of suitable top sheet materials include nonwoven spun-bonded or carded webs of polypropylene, polyethylene, nylon, polyester and blends of these materials, perforated, apertured, or reticulated films, and the like. Nonwoven materials are exemplary because such materials readily allow the passage of liquids to the underlying absorbent laminate core 28. The top sheet 24 preferably comprises a single-ply nonwoven material that may be made of carded fibers, either adhesively or thermally bonded, spunbonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 24, and other top sheet materials will be readily apparent to those skilled in the art.

The top sheet 24 may further comprise several regions having different properties. In one embodiment of the present invention, the laterally distal portions of the top sheet 24, especially those used to make second and third top sheet panels 302, 303, preferably are substantially fluid impervious and hydrophobic, while the remainder of the top sheet 24 (e.g., central top sheet panel 301) is hydrophilic and fluid pervious. Different top sheet properties, such as fluid perviousness and hydrophobicity, may be imparted upon the top sheet 24 by treating the top sheet 24 with adhesives, surfactants, or other chemicals, using a composite of different materials, or by other means. The top sheet 24 may also be made from a laminate of overlaid sheets of material. The top sheet 24 also may be treated in specific areas like the crotch region, with skin wellness ingredients such as aloe, vitamin E, and the like.

As noted elsewhere herein, the top sheet 24 and back sheet 26 may be substantially coterminous, or they may have different shapes and sizes. The particular design of the top sheet 24 and back sheet 26 may be dictated by manufacturing considerations, cost considerations, and performance considerations. Preferably, the top sheet 24 is large enough to completely cover the absorbent laminate core 28, and the back sheet 26 is large enough to prevent leakage from the garment 10. The design of top sheet 24 and back sheet 26 is known in the art, and a skilled artisan will be able to produce an appropriate top sheet 24 and an appropriate back sheet 26 without undue experimentation.

The top sheet 24 and the back sheet 26 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the top sheet 24 to the back sheet 26. The particular joining method may be dictated by the types of materials selected for the top sheet 24 and back sheet 26.

As mentioned above, absorbent garment preferably is provided with leg elastics 30 extending through crotch region 16, adjacent crotch edge 22. The absorbent garment of the invention also preferably is provided with waist elastic material 32 optionally in the first and second waist regions, 12, 14, respectively, to enable and assist in stretching around the wearer. The waist elastics 32 may be similar structures or different to impart similar or different elastic characteristics to the first and second waist regions 12, 14 of the garment. In general, the waist elastics may preferably comprise foam strips positioned at the first and second waist regions 12, 14, respectively. Such foam strips preferably are about ½ to about 1½ inches wide and about 3-6 inches long. The foam strips preferably are positioned between the top sheet portions 24 or panels (301, 302, 303) and the back sheet 26. Alternatively, a plurality of elastic strands may be employed as waist elastics rather than foam strips. The foam strips preferably are comprised of polyurethane, but can be any other suitable material that decreases waist band roll over, reduces leakage over the waist ends of the absorbent garment, and generally improve comfort and fit. The first and optional second waist foam strips 32 preferably are stretched 50-150%, preferably 100% more than their unstretched dimension before being adhesively secured between the back sheet 26 and top sheet 24.

Each edge 22 that forms the leg openings preferably is provided with an adjacent leg elastic containment system 30. In the preferred embodiment, three strands of elastic threads (only two strands are shown in FIG. 2 for purposes of clarity) are positioned to extend adjacent to leg openings between the outer top sheet portions or panels 302, 303 and the back sheet 26. Any suitable elastomeric material exhibiting at least an elongation (defined herein as (L_{S}−L_{R})/L_{R} where L_{S} is the stretch length of an elastic element and L_{R} is retracted length, multiplied by 100 to obtain percent elongation) in the range of 5%-350%, preferably in the range of 200%-300%, can be employed for the leg elastics 30. The leg elastics 30 may be attached to the absorbent article 10 in any of several ways which are known in the art. For example, the leg elastics 30 may be ultrasonically bonded, heat/pressure sealed using a variety of bonding patterns, or glued to the garment 10. Various commercially available materials can be used for the leg elastics 30, such as natural rubber, butyl rubber or other synthetic rubber, urethane, elastomeric materials such as LYCRA (DuPont), GLOSPAN (Globe) or SYSTEM 7000 (Fulflex).

The fastening elements, preferably a fastening system 34 (e.g., tab 34) of the preferred embodiment, is attached to the first waist region 12, and it preferably comprises a tape tab or mechanical fasteners 36. However, any fastening mechanism known in the art will be acceptable. Moreover, the fastening system 34 may include a reinforcement patch below the front waist portion so that the diaper may be checked for soiling without compromising the ability to reuse the fastener. Alternatively, other absorbent article fastening systems are also possible, including safety pins, buttons, and snaps.

As stated previously, the invention has been described in connection with a diaper. The invention, however, is not intended to be limited to application only in diapers. Specifically, the absorbent laminate cores of the preferred embodiments may be readily adapted for use in other absorbent garments besides diapers, including, but not limited to, training pants, feminine hygiene products and adult incontinence products.

The underlying structure beneath the top sheet 24 may include, depending on the diaper construction, various combinations of elements, but in each embodiment, it is contemplated that the absorbent garment will preferably include a multi-component absorbent core composite 28 that contains at least two absorbent core units 286, 288. In addition, an additional layer 290 may be disposed between the top sheet 24 and absorbent core 28, as shown in FIG. 2, and/or other additional layers may be disposed between these layers, or between absorbent core 28 and back sheet 26. An additional layer 290 also may be included in the multi-component absorbent core composite 28. The additional layer(s) 290 may include a fluid transfer layer, a fluid handling layer, a storage layer, a wicking layer, a fluid distribution layer, and any other layer(s) known to those having ordinary skill in the art.

Although the multi-component absorbent core composite 28 depicted in FIG. 2 has a substantially rectangular cross-sectional and plan view shape, other shapes may be used, such as a “T” shape or an hourglass shape. The shape of the multi-component absorbent core composite 28 may be selected to provide the greatest absorbency with a reduced amount of material. The absorbent core may be associated with the top sheet 24, back sheet 26, or any other suitable part of the garment 10 by any method known in the art, in order to fix the multi-component absorbent core composite 28 in place. In addition to the respective layers in the multi-component absorbent core composite 28, as will be described in greater detail hereinafter, the overall multi-component absorbent core composite 28 may be enclosed within a tissue wrapping, as disclosed in U.S. Pat. No. 6,068,620, the disclosure of which is incorporated by reference herein in its entirety. Skilled artisans are capable of designing and wrapping a suitable multi-component absorbent core composite 28 of the invention, using the guidelines provided herein.

The multi-component absorbent core composite 28 may extend into either or both of the first and second waist regions 12, 14. The multi-component absorbent core composite 28 of one preferred embodiment of the invention preferably includes at least two absorbent core units 286, 288, each having at least three (3) layers whereby two of the layers are outer layers, (280, 282, 280′, 282′, FIG. 2), preferably outer tissue layers 280, 282, 280′, 282′ and an inner central layer 284, 284′ one of which contains tow fibers and SAP.

Upper layers 280, 280′ and lower layers 282, 282′ (hereinafter referred to collectively as upper layer 280 and lower layer 282) can be made of any suitable material capable of containing the inner layer(s) of absorbent core 28. Preferably, upper layer 280 is hydrophilic and fluid pervious, and lower layer 282 is hydrophobic and fluid impervious. More preferably, upper layer 280 and lower layer 282 are comprised of the same tissue-like material. It is contemplated in the present invention that absorbent core unit 286 include upper and lower layers 280, 282, but absorbent core unit 288 not contain such an arrangement of materials. Various absorbent core units 288 used in conjunction with absorbent core unit 286 are discussed in more detail below.

In a preferred embodiment, the central layer 284 of absorbent core unit 286 comprises super absorbent polymer distributed within a fibrous structure. The central fibrous layer 284′ of the at least one additional absorbent core unit 288 may be made of any number of individual components, as will be discussed in greater detail below. Central fibrous layers 284, 284′ of this type generally are known in the art, and exemplary absorbent cores are described in U.S. Pat. No. 6,068,620 and U.S. Pat. No. 5,281,207, both issued to Chmielewski, and U.S. Pat. No. 5,863,288, issued to Baker, the disclosures of each of which are herein incorporated by reference in their entirety and in a manner consistent with this disclosure.

Certain fibrous and particulate additives preferably are used as constituent elements of the absorbent core unit 286 to maintain high SAP efficiencies when the SAP concentration is in the range of about 50-95%, more preferably about 60-90%, and most preferably about 75-85%. Super absorbent polymers of the surface cross-linked variety perform best in these laminates. These additives preferably are constituent elements of the central fibrous layer 284, and they may be added to the additional layer(s) 290.

The fibrous component of the central layer 284 of absorbent core unit 286 is comprised of tow fiber, and most preferably is a crimped tow of cellulose acetate or polyester. Before making the absorbent core unit that includes a tow fiber, the tow fiber typically is unwound and opened, and then cut at various lengths to provide a fibrous mass of material. Skilled artisans are aware of techniques available to open tow fibers and form the opened fibers into a fibrous mass.

The fibrous component, if one exists, in central layer 284′ of the at least one second absorbent core unit 288, may be the same or another tow fiber, or it may be a low-density roll good made in a separate process. Still further yet, the fibrous component could also be a carded web formed online. Optionally, it is advantageous to introduce from about 1-5% of a thermally bondable fiber into the fibrous component of the central layer 284 for wet strength and core stability in use.

To maintain high SAP concentrations, the concentration of fibrous material in the central layer 284, 284′ of the absorbent core unit 286, 288 of the invention preferably is about 5-50%, more preferably about 10-30%, and most preferably about 15-25%. Most preferably, the central layer 284 comprises from about 75-85% SAP and from about 15-25% fibrous additives selected from the foregoing group.

Particulate additives may be added to central layer 284 in addition to or as a substitute for the foregoing fibrous additives in order to maintain high SAP efficiency. The particulate additives preferably are insoluble, hydrophilic polymers with particle diameters of 100 μm or less. The particulate additives are chosen to impart optimal separation of the SAP particles. Examples of preferred particulate additive materials include, but are not limited to, potato, corn, wheat, and rice starches. Partially cooked or chemically modified (i.e., modifying hydrophobicity, hydrophilicity, softness, and hardness) starches can also be effective. Most preferably, the particulate additives comprise partially cooked corn or wheat starch because in this state, the corn or wheat are rendered larger than uncooked starch and even in the cooked state remain harder than even swollen SAP. In any event, regardless of the particulate additive chosen, one of the many important criteria is to use particulate additives that are hard hydrophilic materials relative to swollen SAP or which are organic or inorganic polymeric materials about 100 microns in diameter. Fibrous and particulate additives can be used together in these absorbent laminates. Examples of SAP/particulate and SAP/fiber/particulate additives include those described in, for example, U.S. Pat. No. 6,068,620.

Any superabsorbent polymer (SAP) now known or later discovered may be used in central layer 284, or central layer 284′ so long as it is capable of absorbing liquids. Useful SAP materials are those that generally are water-insoluble but water-swellable polymeric substance capable of absorbing water in an amount that is at least ten times the weight of the substance in its dry form. In one type of SAP, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Examples of suitable SAP are water swellable polymers of water soluble acrylic or vinyl monomers crosslinked with a polyfunctional reactant. Also included are starch modified polyacrylic acids and hydrolyzed polyacrylonitrile and their alkali metal salts. A more detailed recitation of superabsorbent polymers is found in U.S. Pat. No. 4,990,541 to Nielsen, the disclosure of which is incorporated herein by reference in its entirety.

Commercially available SAPs include a starch modified superabsorbent polymer available under the tradename SANWET® from Hoechst Celanese Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; FAVOR manufactured by Stockhausen Inc.; AQUA KEEP SA60S, manufactured by Seitetsu Kagaku Co., Ltd.; DIAWET, commercially available from Mitsubishi Chemicals, Japan; FLOSORB, available from SNF Floerger, France, AQUALIC, available from Nippon Shokubai, Osaka, Japan.

In accordance with the present invention, at least one of the absorbent core units is advantageously based upon a tow fiber, and preferably, a continuous crimped filament tow. Accordingly, the central layer 284 is advantageously prepared there from. This fiber structure has high structural integrity, and as such, is distinct from a matrix of discontinuous fibers described as fluff in the prior art. The high structural integrity enables the production of stronger webs than those formed from discontinuous fibers, which in turn are believed to enable the production of thinner absorbent pads. In addition, the use of such fibers enables the production of ultra low density absorbent cores, when compared to absorbent cores prepared by dispersing SAP particles in fluff.

The tow fiber can be any continuous or discontinuous thermoplastic filament tow fiber that is capable of being opened and used in combination with SAP in an absorbent core. Preferably, cellulose ester tow is used as the fibrous material in central layer 284. Non-limiting examples of suitable cellulose esters include cellulose acetate, cellulose propionate, cellulose butyrate, cellulose caproate, cellulose caprylate, cellulose stearate, highly acetylated derivatives thereof such as cellulose diacetate, cellulose triacetate and cellulose tricaproate, and mixtures thereof such as cellulose acetate butyrate. A suitable cellulose ester will include the ability to absorb moisture, preferably is biodegradable, and is influenced not only by the substituent groups but also by the degree of substitution. The relationship between substituent groups, degree of substitution and biodegradability is discussed in W. G. Glasser et al, BIOTECHNOLOGY PROGRESS, vol. 10, pp. 214-219 (1994), the disclosure of which is incorporated herein by reference in its entirety.

Continuous filament tow useful in the present invention is beneficially moisture-absorbent and biodegradable. Accordingly, cellulose acetate tow is typically preferred for use in the invention. Typically, the denier per fiber (dpf) of the tow fiber will be in the range of about 1 to 9, preferably about 3 to 6. For the same weight product, filaments of lower dpf may provide increased surface area and increased moisture absorption. Total denier may vary within the range of about 20,000 to 60,000, depending upon the process used.

It is particularly preferred in the invention to use tow having crimped filaments. Tow materials having crimped filaments are typically easier to open. Separation of filaments resulting from bloom advantageously results in increased available filament surface area for superabsorbent material immobilization and increased moisture absorption. Gel blocking also may be reduced by using crimped tow in the central layer 284. As therefore may be understood, more crimp is typically better, with in excess of about 20 crimps per inch being usually preferred. Continuous filament, cellulose ester tow having crimped filaments with about 25 to 40 crimps per inch, is commercially available from Celanese Acetate in Charlotte, N.C.

If desired, a superabsorbent, absorptive pad of multiple layer thickness, may be provided. To this end, the tow may be, for example, lapped or crosslapped in accordance with conventional procedures. In this way, a superabsorbent, absorptive material of a desired weight and/or thickness may be provided. The specific weight or thickness will depend upon factors including the particular end use. It is especially preferred that the crimped cellulose acetate tow material be opened and then mixed with the SAP particles to form the at least one absorbent core unit.

The SAP may be provided in any particle size, and suitable particle sizes vary greatly depending on the ultimate properties desired. Preferably, a fine particulate rather than a coarse particulate, is used in the invention, and preferably a fine particulate that passes through an about 200 mesh screen is used.

It has been known to prepare absorbent cores comprised of cellulose acetate tow or other polymeric fibers and SAP, as described in H1565, and U.S. Pat. Nos. 5,436,066, and 5,350,370, the disclosures of each of which are incorporated by reference herein in its entirety. It was conventional to add tackifying agents, specific size fibers, or specific fibers in combination with fluff, in order to prepare the absorbent core and immobilize the SAP particles. Use of these materials can make it difficult to vary the properties of the absorbent core throughout its cross-sectional area, but the invention should not be construed to preclude their use. The present invention therefore makes use of this type of core as only one unit of a multi-component absorbent core composite to enable variations in properties of the core throughout its width, length, and thickness.

The total basis weights of the absorbent core unit 286 including fibrous materials, SAP, tissue, additional layers, and additives, are anywhere from about 200-800 grams per square meter. The most preferred total basis weights of the absorbent core unit 286 are about 500-700 grams per square meter. Optionally, about 1-10%, preferably about 5%, by weight of thermally bondable synthetic fibers can be added to the absorbent core 286 to impart additional wet strength to the laminate. This will improve the stability of the core during use of the diaper. The preferred synthetic fibers are polyolefin/polyester fibers and polyester/polyester bicomponent fibers.

Depending on whether a wet or dry process is used to make the absorbent core unit 286, bonding central fibrous layer 284 with any additional layer(s) 290, and tissue layers 280, 282, can be achieved with hydrogen or adhesive bonds. If the material used to form the absorbent core unit 286 contains about 1-5% by weight thermally bondable synthetic fibers, bonding can be achieved with thermal bonds.

The multi-component absorbent core composites 28 of the invention include, in addition to the absorbent core unit 286 discussed above, at least one other absorbent core unit 288 having properties different from absorbent core unit 286. Absorbent core unit 288, and any other additional absorbent core units added to multi-component absorbent core composite 28, may be made from, and made in a similar manner to absorbent core unit 286. The particular amounts of materials may be varied, however, to render the properties of the two core units, 286, 288, different from one another. For example, the amount of SAP employed in absorbent core unit 286 may be different from the amount of SAP employed in absorbent core unit 288. Other variations exist, such as varying the type and amount of adhesive used, varying the basis weights of the respective core units, adding an additional layer(s) 290 to one of the core units, adding a different SAP, fibrous material, additive, etc., and combinations and mixtures of these, as well as any other variations now known or later discovered.

Absorbent core unit 288 may include as a fibrous component, a material other than, or in addition to, the tow fiber used as the fibrous component in absorbent core unit 286. Suitable fibrous components include, but are not limited to, cellulose acetate fibers, rayon fibers, Courtauld's LYOCELL fibers, polyacrylonitrile fibers, surface-modified (hydrophilic) polyester fibers, surface-modified polyolefin/polyester bicomponent fibers, surface-modified polyester/polyester bicomponent fibers, cotton fibers, cottone linters, or blends and/or mixtures thereof. Rayon, Courtauld's LYOCELL, polyacrylonitrile, cotton fibers and cotton linters have similar properties to cellulose acetate and may be used as a fibrous component, if used, in absorbent core unit 288. The remaining fibers, surface-modified polyolefin/polyester bicomponent fibers, and surface-modified polyester/polyester bicomponent fibers are also believed to be effective fibrous additives.

Absorbent core unit 288 also may or may not incorporate outer layers 280′, 282′. Indeed, absorbent core units 286, 288 may be manufactured using either a wet or dry process, or both, and then the two core units disposed in any manner with respect to one another, and ultimately covered with one complete outer layer(s) 280, 282 (designated by numeral 285 in FIG. 3). In this embodiment, outer layer 285 will cover the entire multi-component absorbent core composite 28, which includes at least two absorbent core units 286, 288. Those skilled in the art will appreciate that numerous modifications may be made to the invention while still including two absorbent core units 286, 288 in accordance with the guidelines provided herein.

Another configuration is illustrated in FIG. 3, which includes two separate absorbent core units 286, 288, each containing outer layers 280, 282, 280′, 282′, which then in turn are covered by a cumulative outer layer wrap 285, which may be made of the same or similar material to that used as outer layers 280, 282. As shown in FIG. 3, absorbent core unit 286 includes outer layers 280, 282, and central layer 284. Central layer 284 includes a mixture of tow fiber material (indicated by lines in core 286), and SAP (indicated by black dots in core 286). There are two absorbent core units 286 shown in FIG. 3, surrounding absorbent core unit 288.

Absorbent core unit 288 includes outer layers 280′ and 282′, and central layer 284′. In the embodiment illustrated, central layer 284′ includes only SAP particles, and not fibrous material. The SAP particles of absorbent core unit 288 are indicated by the circles with the horizontal lines through them in the embodiment. The SAP particles in absorbent core unit 288 may be the same as or different from the SAP particles in absorbent core unit 286. It will become apparent to those skilled in the art that using the configuration of FIG. 3, it is possible to utilize a highly efficient and expensive SAP material (e.g., “super-SAP”) only in absorbent core unit 288, and not in the entire absorbent core composite 28, thereby saving money. In addition, the absorbent core unit 288 need not contain any fibrous material in central layer 284, and consequently, the SAP particles can settle to the bottom of the core where they are needed most. The present invention therefore enables the use of very expensive SAP material only in the central portion of the core, where it is needed most.

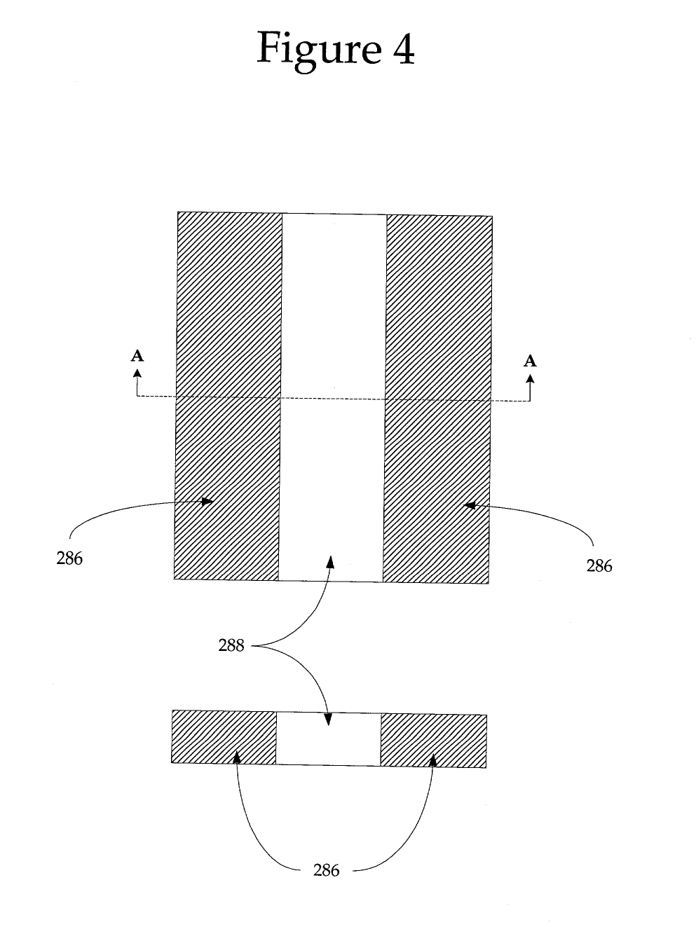

As mentioned above, the possible permutations of the invention are legion. The respective absorbent core units may or may not be coextensive with one another, portions of one core unit may overlap or be disposed above or below another core unit, there may be any number of additional core units, there may be any number of additional layers added to the core unit, etc. The features of the invention therefore provide substantial design flexibility in designing absorbent articles that include the inventive multi-component absorbent core composite 28. A few examples of suitable multi-component absorbent core composites 28 for use in various embodiments of the invention are illustrated in FIGS. 4-13.

One of the preferred features of the invention is that the multi-component absorbent core composite 28 be comprised of two absorbent core units having different properties. The particular properties that are different between the respective core units are not critical to the invention; rather, the skilled artisan is free to select from a vast array of differing properties to design a suitable multi-component absorbent core composite 28 for use in an absorbent material. One potential difference between the absorbent core units (e.g., 286, 288) includes the type and/or properties of the SAP. The properties of the SAP may differ, where various properties include particle size, particle size distribution, capacity, AUL, gel strength, permeability, chemical make-up, processing conditions, degree of cross-linking and modifications, to name but a few.

The ratio of fibrous material to SAP in central layer 284, 284′ also may be varied. It is believed that higher SAP:fiber ratios provide a higher SAP basis weight and thus, a higher overall core capacity. Lower SAP:fiber ratios can provide an absorbent core unit 286, 288 having higher wicking properties. Thus, an absorbent core unit 288 having a relatively low SAP:fiber ratio could be disposed in the center region of the multi-component absorbent core composite 28, and above a larger absorbent core unit 286 having a relatively high SAP:fiber ratio. Numerous other possibilities exist in the context of the present invention.

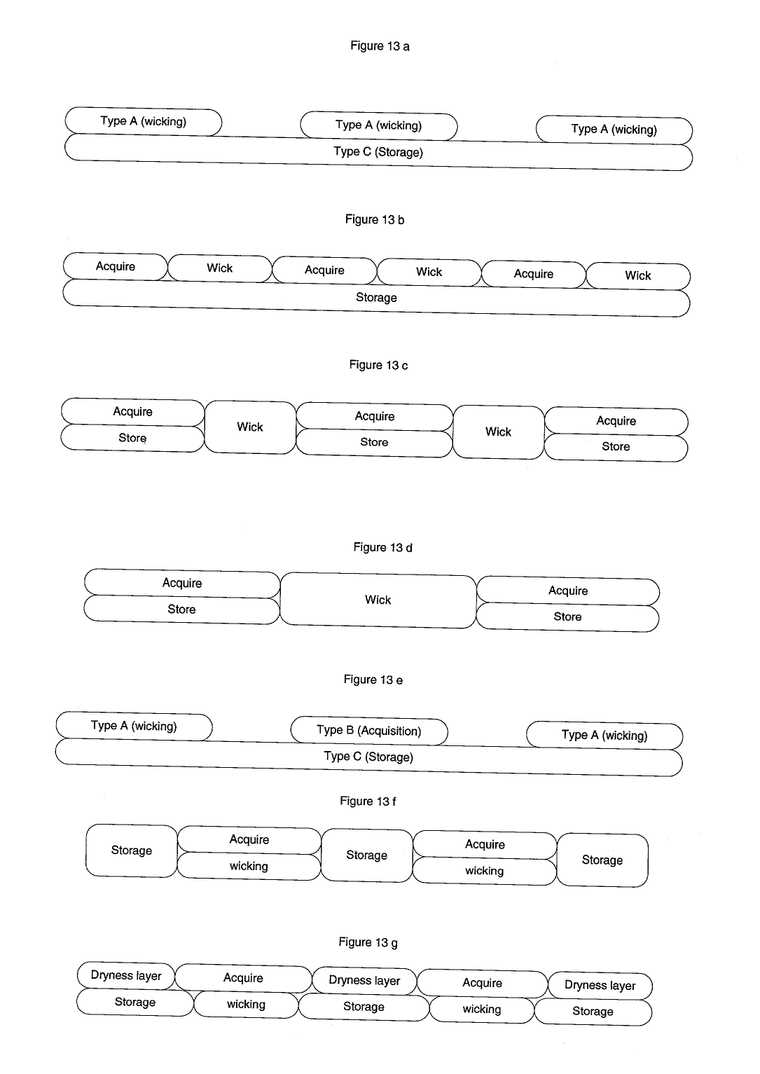

Absorbent core units can be made with high wicking properties without the use of SAP so the pores remain open, which enables wicking to continue regardless of how fluid much the SAP has absorbed (which typically closes off pores as the SAP swells). Acquisition core units can have large pore sizes for high flow because the wicking (which requires small pores) is done in a different core component. Dryness core units or layers also can be used. Dryness layers typically require low hydrophilicity so they stay dry but need to be open structures so they let fluid through, and do not wick fluid (no capillary forces in a hydrophobic layer). This is fine because the other properties typically are handled in the other core components. A variety of configurations of these various layers and core units are shown in FIGS. 13a-13g.

The width of the absorbent core unit also may be varied. A narrower, highly absorbent material can be used in the center portion of the absorbent garment, and a wider less absorbent material used on the outer edges of the multi-component absorbent core composite 28. Adhesives often are employed in an effort to immobilize the SAP particles within the fibrous matrix. The adhesives can be applied to the fibrous material, or it can be applied to outer layers 280, 282. Lower levels of adhesives typically increase the percentage of mobile SAP particles. Thus, a potential multi-component absorbent core composite 28 design might include a central absorbent core unit 288 that has no adhesives (FIG. 3) such that the SAP particles in central layer 284′ are free to settle by gravity or other force to the bottom of the absorbent core unit 288, and an additional absorbent core unit(s) 286 that has a higher amount of adhesive, or that utilizes a fibrous component to entrap the SAP particles.

Other possibilities include varying the absorbent core units 286, 288, etc., by employing additional layers in the absorbent core unit. For example, wicking layers, fluid transfer layers, fluid acquisition and fluid distribution (or redistribution) layers, storage layers, and any other additional layer may be present in one or more of the absorbent core units. The overall basis weights of the absorbent core units 286, 288, etc. may be varied by varying the amount and/or type of fibrous material, amount and/or type of SAP, and the like. If the absorbent core unit 286, 288, etc. employs tissue layers (e.g., upper and lower layers 280, 282 (280′, 282′)) the type of tissue surrounding the respective absorbent core units may be varied. For example, one absorbent core unit may employ a higher wicking tissue layer than another absorbent core unit.

The type and/or amount of fibrous material utilized in the absorbent core unit also may vary. In this regard, one absorbent core unit 286 may include a tow fibrous component, and another absorbent core unit 288 may include a different fibrous component, or a different type of tow material. For example, absorbent core unit 286 may include cellulose ester tow as the fibrous component of central layer 284, whereas absorbent core unit 288 includes a more expensive fiber, such as an Eastman deep grooved wicking fiber in central layer 284′. Skilled artisans will appreciate that there are myriad permutations available for use in designing a suitable multi-component absorbent core composite 28. Using the guidelines provided herein, those skilled in the art are capable of designing a suitable multi-component absorbent core composite 28 for use in an absorbent garment, without undue experimentation.

In the foregoing description of preferred embodiments, absorbent core unit 286 typically is designated as the unit that contains the tow material and SAP, (e.g., first absorbent core unit) and absorbent core unit(s) 288 is/are the at least one additional unit (e.g., second absorbent core unit). These designations are made merely for illustrative purposes only. In the foregoing description and figures, and in the following description of FIGS. 4-10, either absorbent core unit 286 or 288 may be the first absorbent core unit that contains the tow material and SAP, the other(s) be the second absorbent core unit(s) with the different properties. For example, absorbent core unit 288 in FIG. 4 may be the absorbent core unit that contains the tow material and SAP.

FIG. 4 illustrates a possible configuration for the multi-component absorbent core composite 28. In FIGS. 4-9, The figure at the top of the page is a top view of the multi-component absorbent core composite 28, and the figure at the bottom of the page is a cross-sectional view along line A-A. As shown in FIG. 4, the multi-component absorbent core composite 28 includes three separate absorbent core units, two of the same or similar units 286, disposed on the outer perimeter of the core 28, and central unit 288, which is different from the two outer units 286. An embodiment similar to that shown in FIG. 3 would be useful in the configuration shown in FIG. 4. In this regard, absorbent core unit 288 could include a very expensive SAP material, or a different and more expensive type of fiber. Outer absorbent core units 286 need not be made of the same or identical materials, but rather may be comprised of any of a variety of different materials, or the same materials in differing proportions. Numerous other possibilities exist, as will be appreciated by one of ordinary skill in the art.

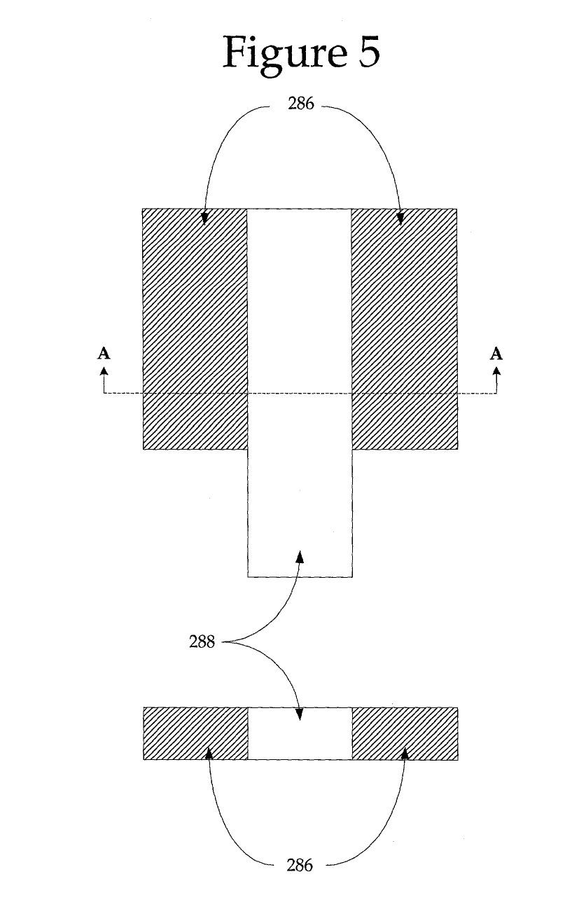

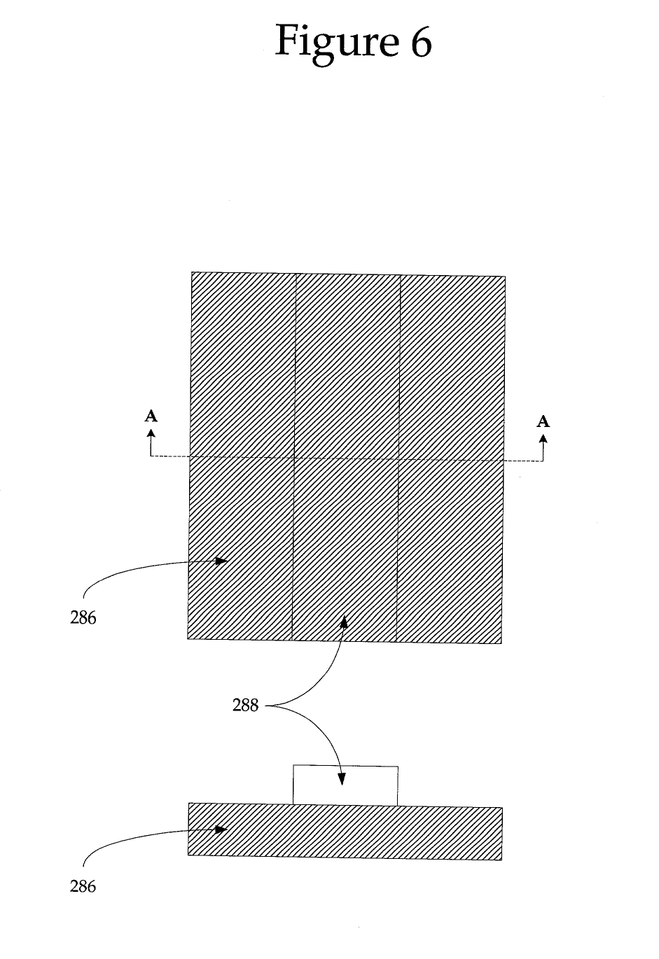

FIG. 5 depicts another possible configuration. Here, a centrally disposed absorbent core unit 288 has a longer dimension in the longitudinal direction 100 (FIG. 1). Such an arrangement would be beneficial where the outer absorbent core units 286 are disposed at or near the front waist region 14 (FIG. 1), where the insult point exists, for crawling or walking babies. This arrangement also would be suitable for newborn babies where the outer absorbent core units 286 are disposed at or near the rear waist region 12 (FIG. 1). The arrangement shown in FIG. 6 is one where the centrally disposed absorbent core unit 288 is on top of absorbent core unit 286. Such an arrangement would be suitable for an absorbent core unit 288 that has better wicking properties than absorbent core unit 286, which has better fluid storage properties. Again, skilled artisans readily recognize that a variety of specifically tailored absorbent core units 286, 288, etc., would benefit from the configuration in any of these figures.

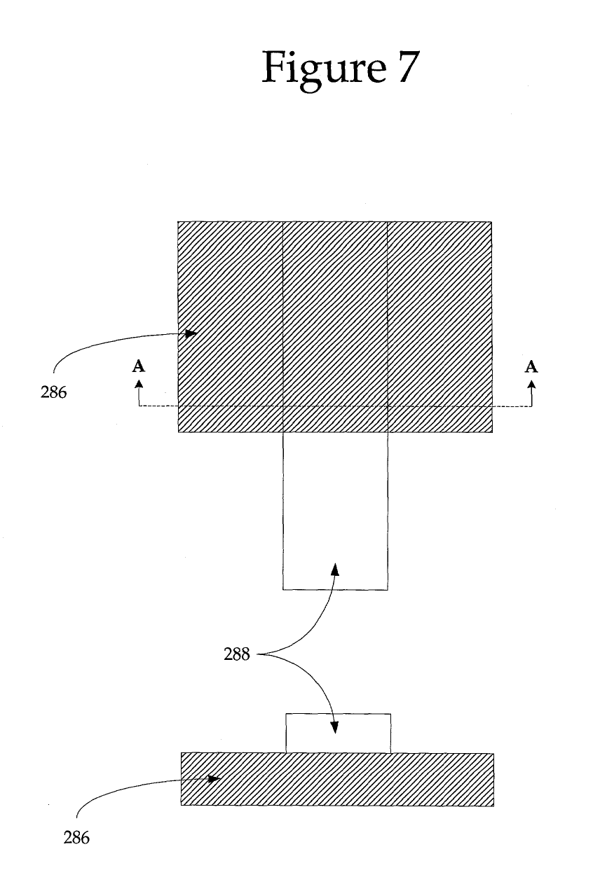

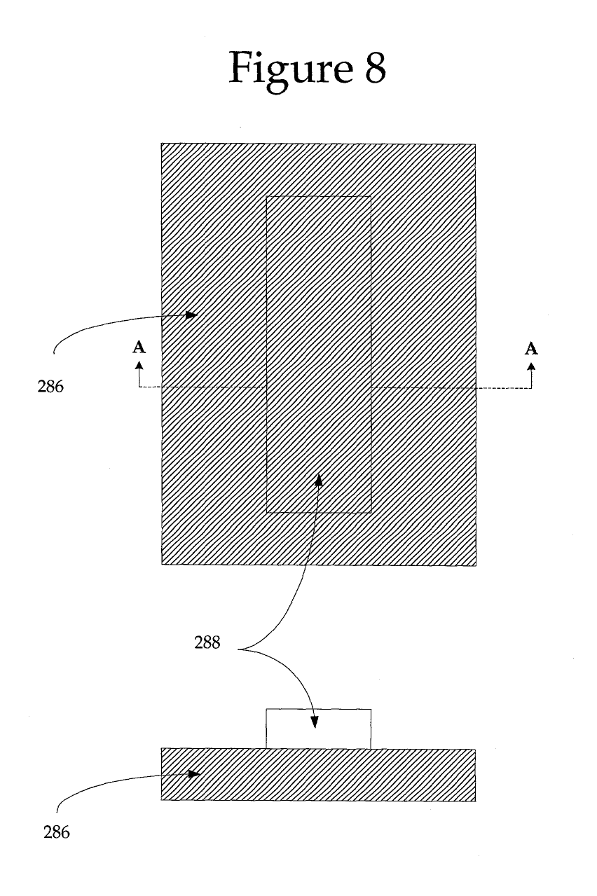

FIG. 7 illustrates another configuration for absorbent core units 286, 288 of the present invention. Centrally disposed absorbent core unit 288 is disposed on top of absorbent core unit 286, and absorbent core unit 286 is disposed at or near one longitudinal end of the core 28. The longitudinal ends of core 28 typically corresponds to first and second waist regions 12, 14, and the longitudinal and lateral center of core 28 corresponds to the crotch region 16. This configuration may be useful for an absorbent core unit 288 that has good longitudinal wicking, and fluid distribution properties, and absorbent core unit 286 that has better fluid storage and acquisition properties. The same holds true for the configuration illustrated in FIG. 8. Here, a centrally disposed absorbent core unit 288 is disposed on top of absorbent core unit 286, which essentially traverses the entire cross-section of the multi-component absorbent core composite 28. Again, skilled artisans readily recognize that a variety of specifically tailored absorbent core units 286, 288, etc., would benefit from the configuration in FIGS. 7 and 8.

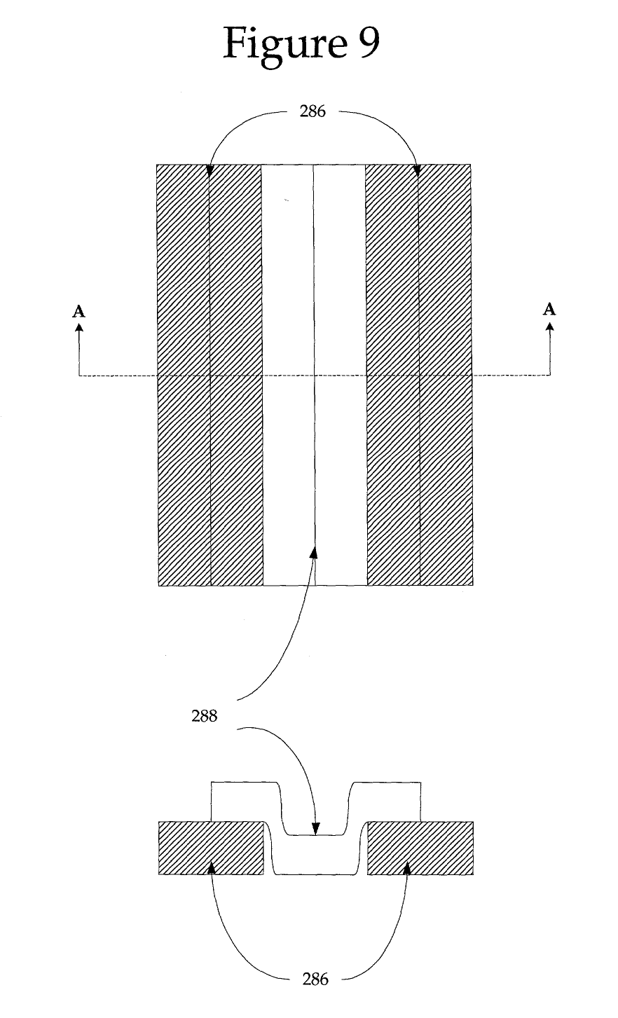

FIG. 9 illustrates yet another configuration of the multi-component absorbent core composite 28. A centrally disposed absorbent core unit 288 is shown between, and above, at least two laterally disposed absorbent core units 286. Again, absorbent core units 286 need not be made of the same or identical materials, but may be comprised of any of a variety of different materials, or the same materials in differing proportions. In the embodiment shown in FIG. 9, centrally disposed absorbent core unit 288 overlaps the laterally disposed absorbent core units 286. The overlapping portions of centrally disposed absorbent core unit 288 are shown above laterally disposed absorbent core units 286, although they may be disposed underneath, as will be appreciated by one of ordinary skill in the art.

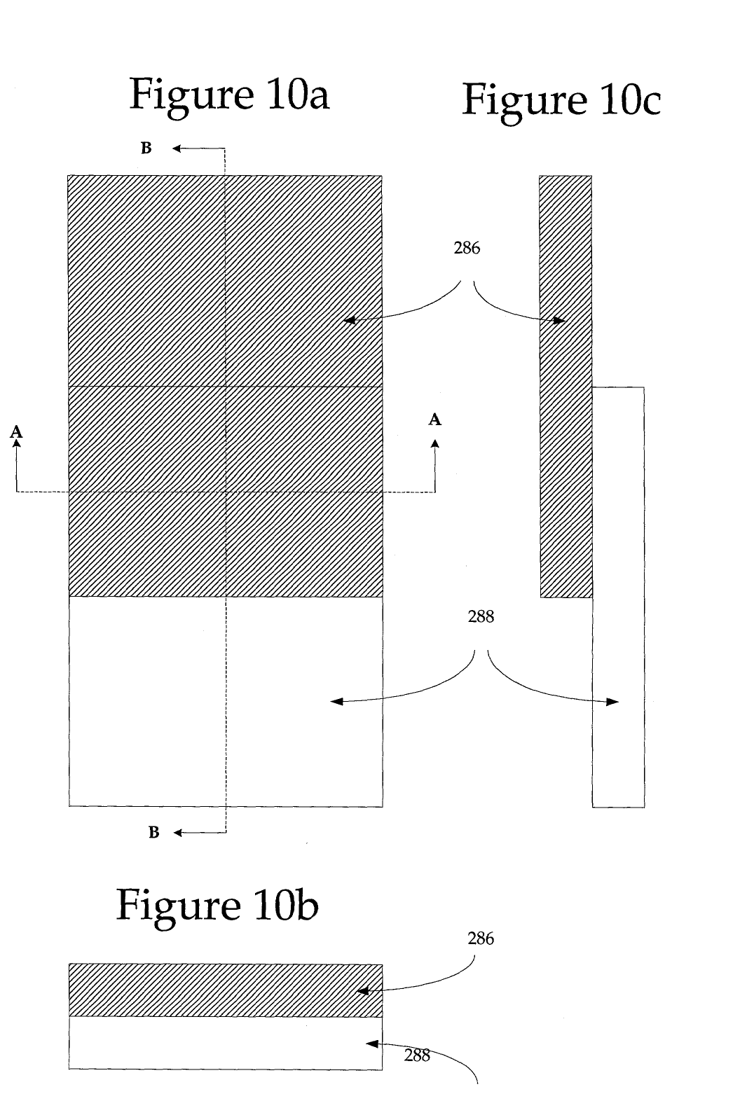

Another configuration of the multi-component absorbent core composite 28 of the invention is illustrated in FIGS. 10a, 10b, and 10c. FIG. 10a depicts a top view of the multi-component absorbent core composite 28, FIG. 10b illustrates a cross-section of the core 28 of FIG. 10a along line A-A, and FIG. 10c shows a cross-section of the core 28 of FIG. 10a along line B-B. As shown therein, one absorbent core unit 286 is disposed near one longitudinal end of multi-component absorbent core composite 28, while the other absorbent core unit 288 is disposed near the longitudinally opposing end of the multi-component absorbent core composite 28. In addition, the respective absorbent core units overlap one another near the longitudinal center of the core 28, with absorbent core unit 288 being disposed on top of the other absorbent core unit 286. Those skilled in the art readily recognize that a variety of specifically tailored absorbent core units 286, 288, etc., would benefit from the configuration in FIGS. 9 and 10.

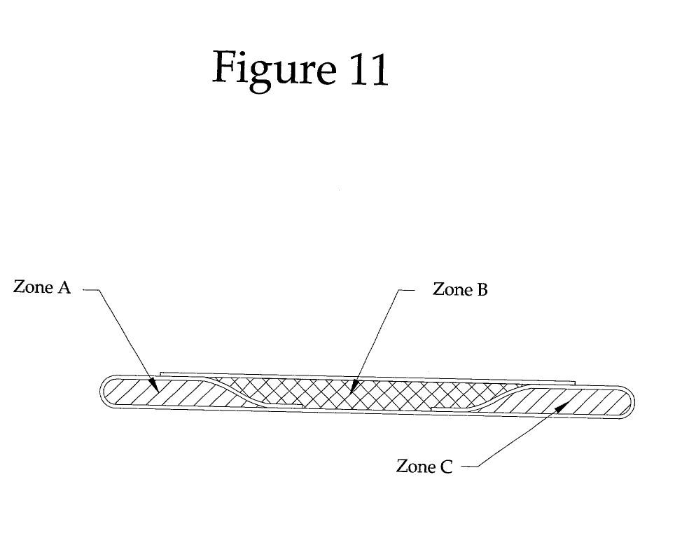

FIG. 11 illustrates a preferred multi-component absorbent core composite of the invention. As shown therein, the outer edges of the absorbent core, zones A and C, can be disposed within a folded core structure. The central portion of the core, zone B, can be disposed outside zones A and C, but within a folded core structure itself. It is believed that the configuration shown in FIG. 11 would be easier to manufacture by virtue of the folded zones.

FIGS. 4-13 represent only a small handful of the possible configurations of multi-component absorbent core composite 28. In addition to other configurations, additional layers may be present in the absorbent core unit, including outer tissue layers. For example, any of the absorbent core units 286,288, etc., may include an additional layer, and/or an additional layer 290 can be disposed outside multi-component absorbent core composite 28, as shown in FIG. 2. Any additional layer can be used, including any layer selected from a fluid acquisition layer, a distribution layer, an additional fibrous layer optionally containing SAP, a wicking layer, a storage layer, or combinations and fragments of these layers. Such layers may be provided to assist with transferring fluids to the multi-component absorbent core composite 28, handling fluid surges, preventing rewet, containing absorbent material, improving core stability, or for other purposes. Skilled artisans are familiar with the various additional layers that may be included in an absorbent article, and the present invention is not intended on being limited to any particular type of materials used for those layers. Rather, the invention encompasses all types of wicking layers, all types of distribution layers, etc., to the extent that type of layer is utilized.

One element that is useful as an additional layer 290 in the absorbent article 10 of the invention is a fluid acquisition layer. The fluid acquisition layer typically comprises a hydrophilic fibrous material, and serves to quickly collect and temporarily hold discharged body fluid. A portion of discharged fluid may, depending upon the wearer's position, permeate the acquisition layer and be absorbed by the central layer 284 in the area proximate to the discharge. However, since fluid is frequently discharged in gushes, the central layer 284 in such area may not absorb the fluid as quickly as it is discharged. Therefore, the fluid acquisition layer hereof also facilitates transport of the fluid from the point of initial fluid contact to other parts of the multi-component absorbent core composite 28. In the context of the present invention, it should be noted that the term “fluid” includes, but is not limited to, liquids, urine, menses, perspiration, and water based body fluids.

The function of the fluid acquisition layer is relatively important. The fluid acquisition layer preferably has sufficient capillary suction to more fully drain the top sheet 24 and yet not exhibit excessive fluid retention to make it difficult for the underlying layer (e.g., central layer 284) to desorb the acquisition layer. The acquisition layer may be comprised of several different materials including nonwoven or woven webs of synthetic fibers including polyester, polypropylene, or polyethylene, natural fibers including cotton or cellulose, blends of such fibers, foams, fluff pulp, apertured films, or any equivalent materials or combinations of materials.

Another useful layer for use in the multi-component absorbent core composite 28 of the invention includes a fluid distribution layer. Fluid distribution layers of the invention can include any combination or all of three basic components: chemically stiffened, twisted, and curled bulking fibers, high surface area fibers, and binder fibers. In a preferred embodiment of the invention, the fluid distribution layer comprises from about 20% to about 80% of the chemically stiffened, twisted, and cured fibers, from about 10% to about 80% of a high surface area fiber, and from 0% to about 50% of a thermoplastic binding means for increasing physical integrity of the web. All percentages herein refer to weight percentages based on total dry web weight. Preferably, the fluid distribution layer will comprise between about 45% and about 60% of chemically stiffened, twisted, and cured fibers, between about 5% and about 15% of a hot melt fibrous binding means, and between about 30% and about 45% high surface area cellulose binding means. More preferably, the fluid distribution layer comprises about 10% thermoplastic binding means, about 45% chemically stiffened, twisted, and cured fibers, and about 45% high surface area fibers.

Chemical additives can also be used as binding means, and are incorporated into the acquisition/distribution layer at levels typically of about 0.2% to about 2.0%, dry web weight basis. The three basic fiber components are described in greater detail in U.S. Pat. No. 5,549,589, the disclosure of which is incorporated by reference herein in its entirety, and in a manner consistent with this disclosure. The fluid distribution layer also may be comprised of non-woven or woven webs of synthetic fibers, natural fibers, foams, carded, thermal bonded materials, and the like.

Another useful layer in the multi-component absorbent core composite 28 of the invention includes a storage layer. Such storage layers typically have limited transport and wicking capabilities but high storage or retention capacity, and rely upon the central layer 284 to distribute incoming fluid over a larger area.

Storage layers or members may be of generally conventional design and composition, selected with regard to the particular application. The storage layer or member may be monolayer or multilayer, homogeneous or stratified, profiled or uniform, etc. Materials suitable for use in such storage layers may be natural or synthetic in origin, woven, non-woven, fibrous, cellular, or particulate, and may include particles, layers, or regions of absorbent polymeric gelling materials. Other preferred materials include fluff pulp and SAP composites, either air laid or wet laid, and high capacity resilient foam materials. Storage layer may also have any desired size and/or shape as may prove suitable for a particular application, including square, rectangular, oval, elliptical, oblong, etc. They may also take on a three-dimensional shape or may be substantially planar in nature.

Another useful layer in multi-component absorbent core composite 28 of the invention is a wicking layer. Wicking layers usually have both fluid acquisition and fluid distribution properties. For example, vertical wicking, which is in general the ability to transport fluids vertically from the top sheet 24 to the multi-component absorbent core composite 28, is related in many respects to fluid acquisition. Horizontal wicking, which is in general the ability to transport fluids along the horizontal 100 and vertical 102 axes of FIG. 1, is related in many respects to fluid distribution.

Any conventional wicking materials can be used for the wicking layer of the invention. High internal phase emulsion (HIPE) foams such as those disclosed in U.S. Pat. No. 5,650,222 can be used, braided materials such as those disclosed in H1,585, and other conventional fibrous and strand materials can be used. The disclosures of U.S. Pat. No. 5,650,222 and H1,585 are incorporated by reference here in their entirety, and in a manner consistent with the present invention.

The wicking layer also may be comprised of two or more sublayers containing absorbent materials with differing wicking characteristics. Any of the materials discussed in this context can be used for any and all of the wicking layers. In accordance with the embodiment of the invention discussed immediately above, the wicking layer may include a first member that is made of a material that is capable of rapidly transferring, in the z-direction (e.g., orthogonal to the plane formed by horizontal 100 and vertical 102 axes of FIG. 1), body fluid that is delivered to top sheet 24. The first member may be designed to have a dimension narrower than the dimension of the multi-component absorbent core composite 28. In this regard, the sides of the first member preferably are spaced away from the longitudinal sides of the multi-component absorbent core composite 28 so that body fluid is restricted to the area within the periphery of the first member, before it passes down and is absorbed into central layer 284 (or second member of the wicking layer). This design is believed to enable the body fluid to be combined in the central area of the multi-component absorbent core composite 28 and to be wicked downward so that a greater quantity of the central layer 284 can be utilized.

A suitable material for use as a first member having high wicking capacity in the z-direction, is a material available from Kimberly-Clark Corporation, in Neenah, Wis. known as PRISM. PRISM is described in U.S. Pat. No. 5,336,552, which is hereby incorporated by reference in its entirety, and in a manner consistent with this disclosure. PRISM generally is a nonwoven fabric and comprises extruded multicomponent polymeric strands including first and second polymeric components arranged in substantially distinctive zones across the cross-section of the multicomponent strands and extending continuously along the length of the multicomponent strands. Preferably, the strands are continuous filaments which may be formed by spunbonding techniques. The second component of the strands constitutes at least a portion of the peripheral surface of the multicomponent strands continuously along the length of the multicomponent strands and includes a blend of a polyolefin and an ethylene alkyl acrylate copolymer. Bonds between the multicomponent strands may be formed by the application of heat.

More specifically, the first polymeric component of the multicomponent strands is present in an amount of from about 20 to about 80 percent by weight of the strands, and the second polymeric component is present in an amount from about 80 to about 20 percent by weight of the strands. Preferably, the first polymeric component of the multicomponent strands is present in an amount of from about 40 to about 60 percent by weight of the strands and the second polymeric component is present in an amount from about 60 to about 40 percent by weight of the strands.

The term “strand” as used herein refers to an elongated extrudate formed by passing a polymer through a forming orifice such as a die. Strands include fibers, which are discontinuous strands having a definite length, and filaments, which are continuous strands of material. The nonwoven fabric of the present invention may be formed from staple multicomponent fibers. Such staple fibers may be carded and bonded to form the nonwoven fabric. Preferably, however, the nonwoven fabric of the present invention is made with continuous spunbond multicomponent filaments which are extruded, drawn and laid on a traveling forming surface.

The types of nonwoven materials that may be employed in any of the wicking layers of the invention include powder-bonded-carded webs, infrared bonded carded webs, and through-air-bonded-carded webs. The infrared and through-air bonded carded webs can optionally include a mixture of different fibers, and the fiber lengths within a selected fabric web may be within the range of about 1.0 to 3.0 inch and an average bulk density of about 0.02 g/cc to about 0.06 g/cc.

The first member of wicking layer also may be a nonwoven fibrous web which includes about 75 percent polyester fibers of at least 6 denier, such as PET (polyethylene terephthalate) fibers available from Celanese AG. The polyester fibers have a length ranging from about 1.5 to 2.0 inches in length. The remaining 25 percent of the fibrous web can be composed of bicomponent binder fibers of not more than 3 denier, and preferably about 1.5 denier. The bicomponent fiber length ranges from about 1.5 to 2 inches. Suitable bicomponent fibers are wettable, polyethylene/polypropylene bicomponent fiber, available from Chisso, a business having offices located in Osaka, Japan. The bicomponent fiber can be a composite, sheath-core type with the polypropylene forming the core and polyethylene forming the sheath of the composite fiber. The polyester fibers and bicomponent fibers generally are homogeneously blended together and are not in a layered configuration. The fibers can be formed into a carded web which is thermally bonded, such as by through-air bonding or infrared bonding.

The second member of wicking layer may be positioned vertically below the first member, and it preferably has a higher wicking capacity along the longitudinal 100 and vertical 102 axes of FIG. 1, than the first member. Preferably, the second member has a wicking capacity at least three times greater than the first member. The second member can be equal in width to the first member, but preferably will be wider. It is preferred that the width of the wicking layer in general be the same as or greater than the width of either central layer 284 if used within an absorbent core unit 286, 288, or the same as or greater than the width of multi-component absorbent core composite 28.

The second member can be a hydrophilic material formed from various types of natural or synthetic fibers including cellulose fibers, surfactant treated meltblown fibers, wood pulp fibers, regenerated cellulose, cotton fibers or a blend of other fibers. Preferably, the second absorbent member is a material described in U.S. Pat. No. 4,100,324, and is generally known as coform. Coform is available from the Kimberly-Clark Corporation located in Neenah, Wis. and is generally a nonwoven material having a fabric-like finish and is made up of an airform matrix of thermoplastic polymeric fibers and a multiplicity of individualized wood pulp fibers. The thermoplastic fiber polymers generally have an average diameter of less than 10 microns with the individualized wood pulp fibers dispersed throughout the matrix and serving to space these microfibers from each other. The material is formed by initially utilizing the primary air stream with the meltblown microfibers and the secondary air stream containing wood pulp fibers and merging the two under turbulent conditions to form an integrated air stream along a forming surface. The fiber-like appearance of this material provides a visual appealing absorbent. Also inherent in the coform material is increased resiliency compared to conventional cellulosic absorbents.

Other suitable materials for use as the wicking layer include high-density air laid fluff pulps, high-density wet laid fluff pulp, and multi-groove fibers such as 4DG deep groove fiber, available from Eastman Chemical Company, Tennessee, or Clemson University, South Carolina.

It is possible in the present invention that the multi-component absorbent core composite 28 be folded as it is disposed between the top sheet 24 and back sheet 26. The multi-component absorbent core composite 28 can be folded in any suitable manner, including any and all of those disclosed in U.S. Pat. No. 6,068,620. Suitable folds include “C” folds, “G” folds, “U” folds, “A” folds, pleats or “W” folds, and the like.

The invention also relates to a method of making an absorbent article that includes providing a top sheet material 24 and a back sheet material 26. The method also includes preparing at least two absorbent core units 286, 288 having different properties, at least one of the absorbent core units being comprised of a mixture of tow fibers and SAP. The method includes arranging the at least two absorbent core units to form a multi-component absorbent core composite 28, and disposing the multi-component absorbent core composite between the top sheet 24 and the back sheet 26. Preparing one of the absorbent core units most preferably includes disposing a central fibrous layer comprising a mixture of tow fibers and SAP between an upper layer and a lower layer.

In accordance with the method, the multi-component absorbent core composite 28 can be arranged in any number of possible configurations. It is a feature of the invention that the multi-component absorbent core composite 28 include at least a first absorbent core unit 286 that includes a mixture of tow fiber material and SAP, and at least one second absorbent core unit 288 that is different from the first absorbent core unit 286. The particular arrangement of first absorbent core unit 286, and the at least one second absorbent core unit 288 is not critical to the invention. The following description includes only a handful of the possible methods of arranging the multi-component absorbent core composite 28 of the invention.

The absorbent article of the invention typically comprises a front waist region 14, a rear waist region 12, and a centrally disposed crotch region 16 (FIG. 1). In one embodiment of the invention, the method includes laterally disposing the first absorbent core unit between at least two second absorbent core units, whereby all of the absorbent core units longitudinally extend from the front waist region, through the crotch region and into the rear waist region (FIGS. 3 and 4). As mentioned earlier with reference to FIGS. 4-10, first and second absorbent core units can be referenced by either numeral 286 or 288 in the drawings. In another embodiment, the method includes laterally disposing the first absorbent core unit between at least two second absorbent core units, and the two second absorbent core units are disposed near one of the waist regions, (e.g., the front or rear waist region) and the first absorbent core unit longitudinally extends from one waist region to the other (FIG. 5).

Another embodiment of the method includes disposing the first absorbent core unit above the second absorbent core unit, in a position near the lateral center of the second absorbent core unit (FIG. 6). The method also may include disposing the first absorbent core unit above the second absorbent core unit, whereby the two second absorbent core units are disposed near one of the waist regions, (e.g., front or rear) and the first absorbent core unit longitudinally extends from one waist region to the other (FIG. 7).

In yet another embodiment of the method of the invention, the method includes disposing the first absorbent core unit above the second absorbent core unit, whereby the first absorbent core unit is disposed in the crotch region, and the at least one second absorbent core unit longitudinally extends from one waist region to the other (FIG. 8). Another method of the invention includes disposing the first absorbent core unit above the second absorbent core, whereby the first absorbent core unit is disposed near one waist region and extends longitudinally into the crotch region, and the at least one second absorbent core unit is disposed near the other waist region and extends longitudinally into the crotch region where it is overlapped by the first absorbent core unit.

The foregoing description of certain features, advantages, and disadvantages of the present invention, and of the known art, is in no way intended to limit the invention. For example, certain embodiments of the invention may be capable of overcoming some of the disadvantages of the prior art, yet still retain some of the embodiments, features, structures, apparatus, etc. of the prior art.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. An absorbent article having a longitudinal dimension and a lateral dimension comprising:
a top sheet;
a back sheet; and
a multi-component absorbent core composite disposed between the top sheet and the back sheet;
wherein the multi-component absorbent core composite includes at least a first and second different absorbent core units, where at least the first absorbent core unit is comprised of a mixture of tow fibers and superabsorbent polymer (SAP).

2. The absorbent article of claim 1, whereby the article has a first waist region, a second waist region longitudinally opposed to the first waist region, and a crotch region between the first and second waist regions, the article further comprising at least one fastening element attached to a lateral edge of the first waist region; and
one or more target devices attached to the article in the second waist region, where at least one fastening element and the one or more target devices are capable of attaching to one another, the one or more target devices being located so that the first waist region and second waist region of the garment may be joined to one another to secure the garment on a wearer.

3. The absorbent article of claim 2, further comprising elastic leg gathers comprising one or more elastic materials disposed adjacent a lateral edge of the crotch region, and standing leg gathers disposed on the top sheet adjacent the lateral edge of the crotch region.

4. The absorbent article of claim 2, wherein the at least one fastening element comprises a hook portion of a hook and loop fastener and the one or more target devices comprise the loop portion of a hook and loop fastener.

5. The absorbent article of claim 2, wherein the at least one fastening element is an adhesive tape and the one or more target devices comprise a tape receiving surface.

6. The absorbent article of claim 2, wherein the at least one fastening element is comprised of a pair of laterally extending tabs disposed on the lateral edges of the first waist region, whereby the laterally extending tabs each include at least one fastening element.

7. The absorbent article of claim 1, wherein at least one additional layer is disposed between the multi-component absorbent core composite and the top sheet.

8. The absorbent article of claim 7, wherein the at least one additional layer is selected from the group consisting of a fluid acquisition layer, a distribution layer, an additional fibrous layer optionally containing SAP, a wicking layer, a storage layer, and combinations and fragments thereof.

9. The absorbent article of claim 1, wherein one of the absorbent core units includes at least one additional layer.

10. The absorbent article of claim 9, wherein the at least one additional layer is selected from the group consisting of a fluid acquisition layer, a distribution layer, an additional fibrous layer optionally containing SAP, a wicking layer, a storage layer, and combinations and fragments thereof.

11. The absorbent article of claim 1, wherein the absorbent core unit that includes tow and SAP is a multi-layered absorbent core unit including two outer tissue layers and a central layer that comprises from about 50% to about 95% by weight super absorbent polymer (SAP), and has a SAP efficiency of at least 80%.

12. The absorbent article of claim 11, wherein the central layer comprises tow fibers selected from the group consisting of cellulose acetate fibers, rayon fibers, LYOCELL fibers, polyacrylonitrile fibers, cotton fibers and cotton linter fibers.

13. The absorbent article of claim 11, wherein the central layer further comprises up to 10% by weight fluff wood pulp fibers.

14. The absorbent article of claim 11, wherein the central layer further comprises particulate additives.

15. The absorbent article of claim 1, wherein the tow is a cellulose ester tow.

16. The absorbent article of claim 15, wherein the tow is a cellulose acetate tow.

17. The absorbent article of claim 1, wherein the at least one second absorbent core unit is comprised of the same materials as the first absorbent core unit in differing amounts.

18. The absorbent article of claim 17, wherein both the first and the at least one second absorbent core unit comprise at least fiber and SAP, and the at least one second absorbent core unit has a different SAP:fiber ratio than the first absorbent core unit.

19. The absorbent article of claim 1, wherein the at least one second absorbent core unit is comprised of at least one different material than the first absorbent core unit.

20. The absorbent article of claim 19, wherein the at least one second absorbent core unit includes a different fibrous component than the first absorbent core unit.

21. The absorbent article of claim 19, wherein the at least one second absorbent core unit does not include a fibrous component.

22. The absorbent article of claim 19, where the at least one second absorbent core unit includes a different SAP than the first absorbent core unit.

23. The absorbent article of claim 19, wherein the at least one second absorbent core unit includes a different amount of adhesive than that used in the first absorbent core unit.

24. The absorbent article of claim 1, wherein the first absorbent core unit is disposed between two second absorbent core units.

25. The absorbent article of claim 24, wherein the absorbent article comprises two waist regions disposed near its longitudinal ends, and a centrally disposed crotch region, whereby the two second absorbent core units are disposed near one of the waist region, and the first absorbent core unit longitudinally extends from one waist region to the other.

26. The absorbent article of claim 1, wherein the first absorbent core unit is disposed above the second absorbent core unit, and is disposed near the middle of the second absorbent core unit.

27. The absorbent article of claim 26, the absorbent article comprises two waist regions disposed near its longitudinal ends, and a centrally disposed crotch region, whereby the two second absorbent core units are disposed near one of the waist region, and the first absorbent core unit longitudinally extends from one waist region to the other.

28. The absorbent article of claim 26, the absorbent article comprises two waist regions disposed near its longitudinal ends, and a centrally disposed crotch region, whereby the first absorbent core unit is disposed in the crotch region, and the at least one second absorbent core unit longitudinally extends from one waist region to the other.

29. The absorbent article of claim 26, the absorbent article comprises two waist regions disposed near its longitudinal ends, and a centrally disposed crotch region, whereby the first absorbent core unit is deposed near one waist region and extends longitudinally into the crotch region, and the at least one second absorbent core unit is disposed near the other waist region and extends longitudinally into the crotch region where it is overlapped by the first absorbent core unit.

30. A method of making an absorbent article comprising:
a) preparing a top sheet and a back sheet;
b) preparing a multi-component absorbent core composite by:
b1) preparing a first absorbent core unit that contains a mixture of tow fibers and SAP;
b2) preparing at least one second absorbent core unit that is different from the first absorbent core unit; and
b3) arranging the first and the at least one second absorbent core units to form a multi-component absorbent core composite; and
c) disposing the multi-component absorbent core composite between the top sheet and the back sheet,

31. The method of claim 30, further comprising disposing at least one additional layer between the multi-component absorbent core composite and the top sheet.

32. The method of claim 31, wherein the at least one additional layer is selected from the group consisting of a fluid acquisition layer, a distribution layer, an additional fibrous layer optionally containing SAP, a wicking layer, a storage layer, and combinations and fragments thereof.

33. The method of claim 30, further comprising adding an additional layer to the first or the at leas one second absorbent core units.

34. The method of claim 33, wherein the at least one additional layer is selected from the group consisting of a fluid acquisition layer, a distribution layer, an additional fibrous layer optionally containing SAP, a wicking layer, a storage layer, and combinations and fragments thereof.

35. The method of claim 30, wherein preparing the first absorbent core unit the absorbent core unit that includes tow and SAP comprises providing two outer tissue layers; and disposing a central layer including at least tow fiber and SAP between the outer tissue layers, whereby the central layer that comprises from about 50% to about 95% by weight super absorbent polymer (SAP), and has a SAP efficiency of at least 80%.

36. The method of claim 35, wherein the central layer comprises tow fibers selected from the group consisting of cellulose acetate fibers, rayon fibers, LYOCELL fibers, polyacrylonitrile fibers, cotton fibers and cotton linter fibers.

37. The method of claim 35, wherein the central layer further comprises up to 10% by weight fluff wood pulp fibers.

38. The method of claim 35, wherein the central layer further comprises particulate additives.

39. The method of claim 35, wherein the tow is a cellulose ester tow.

40. The method of claim 39, wherein the tow is a cellulose acetate tow.

41. The method of claim 30, wherein the at least one second absorbent core unit is comprised of the same materials as the first absorbent core unit in differing amounts.

42. The method of claim 41, wherein both the first and the at least one second absorbent core unit comprise at least fiber and SAP, and the at least one second absorbent core unit has a different SAP:fiber ratio than the first absorbent core unit.

43. The method of claim 30, wherein the at least one second absorbent core unit is comprised of at least one different material than the first absorbent core unit.

44. The method of claim 43, wherein the at least one second absorbent core unit includes a different fibrous component than the first absorbent core unit.

45. The method of claim 43, wherein the at least one second absorbent core unit does not include a fibrous component.

46. The method of claim 43, where the at least one second absorbent core unit includes a different SAP than the first absorbent core unit.

47. The method of claim 43, wherein the at least one second absorbent core unit includes a different amount of adhesive than that used in the first absorbent core unit.

48. The method of claim 30, the method comprising disposing the first absorbent core unit between two second absorbent core units.

49. The method of claim 48, wherein the absorbent article comprises two waist regions disposed near its longitudinal ends, and a centrally disposed crotch region, the method comprising disposing the two second absorbent core units near one of the waist region, and the longitudinally extending the first absorbent core unit from one waist region to the other.

50. The method of claim 30, further comprising disposing the first absorbent core unit above the second absorbent core unit, whereby the first absorbent core unit is disposed near the middle of the second absorbent core unit.

51. The method of claim 50, the absorbent article comprises two waist regions disposed near its longitudinal ends, and a centrally disposed crotch region, the method comprising disposing the two second absorbent core units near one of the waist region, and longitudinally extending the first absorbent core unit from one waist region to the other.

52. The method of claim 50, the absorbent article comprises two waist regions disposed near its longitudinal ends, and a centrally disposed crotch region, the method comprising disposing the first absorbent core unit in the crotch region, and longitudinally extending the at least one second absorbent core unit from one waist region to the other.

53. The method of claim 50, the absorbent article comprises two waist regions disposed near its longitudinal ends, and a centrally disposed crotch region, the method comprising disposing the first absorbent core unit near one waist region and longitudinally extending the first absorbent core unit into the crotch region, and disposing the at least one second absorbent core unit near the other waist region and longitudinally extending the at least one second absorbent core unit into the crotch region where it is overlapped by the first absorbent core unit
